(19) United States
(12) Patent Application Publication
(10) Pub. No.: US 2012/0122820 A1
(43) Pub. Date: May 17, 2012

(54) PRODRUGS AND THE USE THEREOF

(75) Inventors: Nicole Diedrichs, Velbert (DE); Thomas Krahn, Hagen (DE); Ursula Krenz, Leichlingen (DE); Jörg Keldenich, Wuppertal (DE); Hanna Tinel, Wuppertal (DE); Claudia Hirth-Dietrich, Wuppertal (DE); Hans-Georg Lerchen, Leverkusen (DE)

(73) Assignee: Bayer Schering Pharma Aktiengesellschaft

(21) Appl. No.: 12/671,681

(22) PCT Filed: Jul. 23, 2008
(86) PCT No.: PCT/EP2008/006028

§ 371 Date: Jul. 27, 2011

(30) Foreign Application Priority Data
Aug. 1, 2007 (DE) 102007036075.6

(51) Int. Cl. A61K 31/675; A61P 9/00; C07D 417/14; A61K 31/4439; C07F 9/6539; C07D 417/12

(52) U.S. Cl. 514/89; 546/24; 546/269.7; 514/342; 514/341

(57) ABSTRACT

The present application relates to prodrug derivatives of 2-amino-6-({[2-(4-chlorophenyl)-1,3-thiazol-4-yl]methyl}thio)-4-[4-(2-hydroxyethoxy)phenyl]pyridine-3,5-dicarbonitrile, processes for their preparation, their use for the treatment and/or prophylaxis of diseases, and their use for the manufacture of medicaments for the treatment and/or prophylaxis of diseases, especially of cardiovascular disorders.

The present application relates to prodrug derivatives of 2-amino-6-({[2-(4-chlorophenyl)-1,3-thiazol-4-yl]methyl}thio)-4-[4-(2-hydroxyethoxy)phenyl]pyridine-3,5-dicarbonitrile, processes for their preparation, their use for the treatment and/or prophylaxis of diseases, and their use for the manufacture of medicaments for the treatment and/or prophylaxis of diseases, especially of cardiovascular disorders.

Prodrugs are derivatives of an active ingredient which undergo in vivo an enzymatic and/or chemical biotransformation in one or more stages before the actual active ingredient is liberated. A prodrug residue is ordinarily used in order to improve the profile of properties of the underlying active ingredient [P. Ettmayer et al., J. Med. Chem. 47, 2393-2404 (2004)]. In order to achieve an optimal profile of effects it is necessary in this connection for the design of the prodrug residue as well as the desired mechanism of liberation to conform very accurately with the individual active ingredient, the indication, the site of action and the administration route. A large number of medicaments is administered as prodrugs which exhibit an improved bioavailability by comparison with the underlying active ingredient, for example achieved by improving the physicochemical profile, specifically the solubility, the active or passive absorption properties or the tissue-specific distribution. An example which may be mentioned from the wide-ranging literature on prodrugs is: H. Bundgaard (Ed.), Design of Prodrugs: Bioreversible derivatives for various functional groups and chemical entities, Elsevier Science Publishers B.V., 1985.

Adenosine, a purine nucleoside, is present in all cells and is released under a large number of physiological and pathophysiological stimuli. Adenosine is produced inside cells on degradation of adenosine 5′-monophosphate (AMP) and S-adenosylhomocysteine as intermediate, but can be released from the cell and then exerts, by binding to specific receptors, effects as hormone-like substance or neurotransmitter. Essential functions in particular in excitable and/or working cells in various tissues are influenced by adenosine A1 receptors [cf. K. A. Jacobson and Z.-G. Gao, Nat. Rev. Drug Discover. 5, 247-264 (2006)].

The compound 2-amino-6-({[2-(4-chlorophenyl)-1,3-thiazol-4-yl]methyl}thio)-4-[4-(2-hydroxy-ethoxy)phenyl]pyridine-3,5-dicarbonitrile [compound (A)] is an orally active adenosine A1 receptor agonist and is currently undergoing in-depth clinical testing as a possible novel active pharmaceutical ingredient for the prevention and therapy in particular of cardiovascular disorders [WHO Drug Information Vol. 20, No. 2 (2006); for preparation and use, see WO 03/053441, example 6].

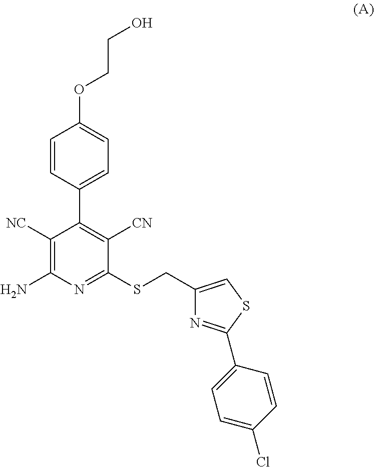

However, compound (A) has only a limited solubility in water, physiological media and organic solvents, and an only low bioavailability after oral administration of a suspension of crystalline material. On the one hand, this allows intravenous administration of the active ingredient only in very low dosages; infusion solutions based on physiological saline solutions can be produced only with difficulty with conventional solubilizers. On the other hand formulation in tablet form is difficult. It was therefore an object of the present invention to identify derivatives or prodrugs of compound (A) which have an improved solubility in the media mentioned and/or an improved bioavailability after oral administration and, at the same time, allow controlled liberation of the active ingredient (A) in the patient's body after administration. In addition, further areas of therapeutic use of this active ingredient could be opened up by an improved possibility of intravenous administration.

A review of prodrug derivatives based on carboxylic esters and possible properties of such compounds is given for example in K. Beaumont et al., Curr. Drug Metab. 4, 461-485 (2003).

The present invention relates to compounds of the general formula (I)

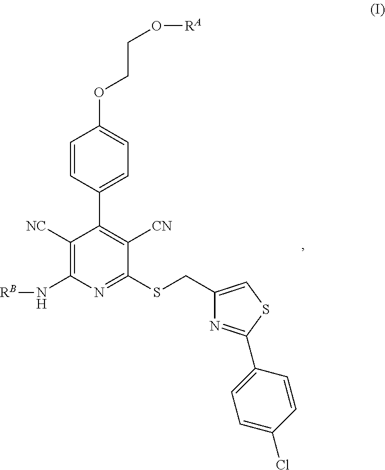

in which
R^{A} is a group of the formula

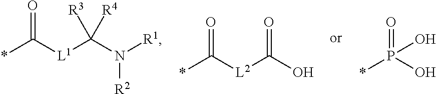

in which
* means the point of linkage to the O atom,
L¹ is a bond, —CH₂— or —CH₂CH₂—,
R¹ and R² are identical or different and are independently of one another hydrogen or (C₁-C₄)-alkyl which may be substituted by hydroxyl, (C₁-C₄)-alkoxy, amino, mono-(C₁-C₄)-alkylamino or di-(C₁-C₄)-alkylamino,
or
R¹ and R² are attached to one another and together with the nitrogen atom to which they are attached form a 5- or 6-membered saturated heterocycle which may contain a further ring heteroatom from the group consisting of N and O and which may be mono- or disubstituted by identical or different substituents from the group consisting of (C₁-C₄)-alkyl, amino, hydroxyl and (C₁-C₄)-alkoxy,
R³ is hydrogen or the side group of a natural α-amino acid or its homologs or isomers
or
R³ is attached to R¹ and the two together with the atoms to which they are attached form a 5- or 6-membered saturated heterocycle which may be mono- or disubstituted by identical or different substituents from the group consisting of (C₁-C₄)-alkyl, amino, hydroxyl and (C₁-C₄)-alkoxy,
R⁴ is hydrogen or methyl,
L² is a bond or straight-chain (C₁-C₆)-alkanediyl or (C₂-C₆)-alkenediyl which may be substituted up to four times by identical or different radicals selected from the group consisting of (C₁-C₄)-alkyl, hydroxyl, (C₁-C₄)-alkoxy, amino, mono-(C₁-C₄)-alkylamino and di-(C₁-C₄)-alkylamino,
   where (C₁-C₄)-alkyl for its part may be substituted by hydroxyl, (C₁-C₄)-alkoxy, amino, mono-(C₁-C₄)-alkylamino or di-(C₁-C₄)-alkylamino
   and/or
   two of the (C₁-C₄)-alkyl radicals mentioned may be attached to one another and together with the carbon atom(s) to which they are attached form a 3- to 6-membered saturated carbocycle which may be substituted by amino, hydroxyl or (C₁-C₄)-alkoxy,
      and
      R^{B} is hydrogen or a group of the formula

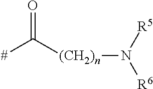

in which
# means the point of linkage to the N atom,
n is the number 1, 2, 3 or 4
and
R⁵ and R⁶ are independently of one another hydrogen or (C₁-C₄)-alkyl, and the salts, solvates and solvates of the salts thereof.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds according to the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

Where the compounds according to the invention can occur in tautomeric forms, the present invention encompasses all tautomeric forms.

Salts preferred for the purposes of the present invention are physiologically acceptable salts of the compounds according to the invention. However, salts which are themselves unsuitable for pharmaceutical applications but can be used for example for isolating or purifying the compounds according to the invention are also encompassed. In addition to mono-salts, the present invention also includes any possible multiple salts, such as di- or tri-salts.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of usual bases such as, by way of example and preferably, alkali metal salts (e.g. sodium and potassium salts), alkaline earth metal salts (e.g. calcium and magnesium salts) and ammonium salts, derived from ammonia or organic amines having 1 to 16 C atoms, such as, by way of example and preferably, ethylamine, diethylamine, triethylamine, ethyldiisopropylamine, monoethanolamine, diethanolamine, triethanolamine, choline, dicyclohexylamine, dimethylaminoethanol, procain, dibenzylamine, morpholine, N-methylmorpholine, arginine, lysine, ethylenediamine, piperidine and N-methylpiperidine.

Solvates refer for the purposes of the invention to those forms of the compounds according to the invention which form a complex in the solid or liquid state through coordination with solvent molecules. Hydrates are a specific form of solvates in which the coordination takes place with water. Solvates preferred in the context of the present invention are hydrates.

In the context of the present invention, the substituents have the following meaning unless otherwise specified:

(C₁-C₄)-Alkyl is in the context of the invention a straight-chain or branched alkyl radical having 1 to 4 carbon atoms. Examples which may be preferably mentioned are: methyl, ethyl, n-propyl, isopropyl, n-butyl, isobutyl, sec-butyl, tert-butyl.

(C₁-C₄)-Alkoxy is in the context of the invention a straight-chain or branched alkoxy radical having 1 to 4 carbon atoms. Examples which may be preferably mentioned are: methoxy, ethoxy, n-propoxy, isopropoxy, n-butoxy, tert-butoxy.

Mono-(C₁-C₄)-alkylamino is in the context of the invention an amino group having a straight-chain or branched alkyl substituent having 1 to 4 carbon atoms. Examples which may be preferably mentioned are: methylamino, ethylamino, n-propylamino, isopropylamino, n-butylamino, tert-butylamino.

Di-(C₁-C₄)-alkylamino is in the context of the invention an amino group having two identical or different straight-chain or branched alkyl substituents having 1 to 4 carbon atoms each.

Examples which may be preferably mentioned are: N,N-dimethylamino, N,N-diethylamino, N-ethyl-N-methylamino, N-methyl-N-n-propylamino, N-isopropyl-N-n-propylamino, N,N-diisopropylamino, N-n-butyl-N-methylamino, N-tert-butyl-N-methylamino.

(C₁-C₆)-Alkanediyl is in the context of the invention a straight-chain divalent alkyl radical having 1 to 6 carbon atoms. A straight-chain alkanediyl radical having 1 to 4 carbon atoms is preferred. Examples which may be preferably mentioned are: methylene, 1,2-ethylene, 1,3-propylene, 1,4-butylene, 1,5-pentylene, 1,6-hexylene.

(C₂-C₆)-Alkenediyl is in the context of the invention a straight-chain divalent alkenyl radical having 2 to 6 carbon atoms and up to 2 double bonds. A straight-chain alkenediyl radical having 2 to 4 carbon atoms and one double bond is preferred. Examples which may be preferably mentioned are: ethene-1,2-diyl, propene-1,3-diyl, but-1-ene-1,4-diyl, but-2-ene-1,4-diyl, buta-1,3-diene-1,4-diyl, pent-2-ene-1,5-diyl, hex-3-ene-1,6-diyl and hexa-2,4-diene-1,6-diyl.

A 3- to 6-membered carbocycle is in the context of the invention a monocyclic saturated cycloalkyl group having 3 to 6 ring carbon atoms. Examples which may be preferably mentioned are: cyclopropyl, cyclobutyl, cyclopentyl, cyclohexyl.

A 5- or 6-membered heterocycle is in the context of the invention a monocyclic saturated heterocycloalkyl group having a total of 5 or 6 ring atoms which contains a ring nitrogen atom and may optionally contain a second ring heteroatom from the group consisting of N and O. Examples which may be preferably mentioned are: pyrrolidinyl, piperidinyl, piperazinyl, morpholinyl.

The side group of an α-amino acid in the meaning of R³ encompasses both the side groups of naturally occurring α-amino acids and the side groups of homologs and isomers of these α-amino acids. The α-amino acid may in this connection have both the L and the D configuration or else be a mixture of the L form and D form. Examples of side groups which may be mentioned are: methyl (alanine), propan-2-yl (valine), propan-1-yl (norvaline), 2-methylpropan-1-yl (leucine), 1-methylpropan-1-yl (isoleucine), butan-1-yl (norleucine), tert-butyl (2-tert-butylglycine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-yl (lysine), 4-amino-3-hydroxybutan-1-yl (hydroxylysine), 3-aminopropan-1-yl (ornithine), 2-aminoethyl (2,4-diaminobutyric acid), aminomethyl (2,3-diaminopropionic acid), 3-guanidinopropan-1-yl (arginine), 3-ureidopropan-1-yl (citrulline). Preferred α-amino acid side groups in the meaning of R⁴ are methyl (alanine), propan-2-yl (valine), propan-1-yl (norvaline), butan-1-yl (norleucine), benzyl (phenylalanine), imidazol-4-ylmethyl (histidine), hydroxymethyl (serine), 1-hydroxyethyl (threonine), 2-carboxyethyl (glutamic acid), 4-aminobutan-1-yl (lysine), 3-aminopropan-1-yl (ornithine), 2-aminoethyl (2,4-diaminobutyric acid), aminomethyl (2,3-diaminopropionic acid), 3-guanidinopropan-1-yl (arginine). The L configuration is preferred in each case.

If radicals in the compounds according to the invention are substituted, the radicals may, unless otherwise specified, be substituted one or more times. In the context of the present invention, all radicals which occur more than once have a mutually independent meaning. Substitution by one or two identical or different substituents is preferred. Substitution by one substituent is very particularly preferred.

Preference is given to compounds of the formula (I) in which

R^{A} is a group of the formula

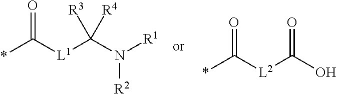

in which
* means the point of linkage to the O atom,
L¹ is a bond, —CH₂— or —CH₂CH₂—,
R¹ and R² are independently of one another hydrogen or methyl
or
R¹ and R² are attached to one another and together with the nitrogen atom to which they are attached form a pyrrolidino, piperidino or morpholino ring,
R³ is hydrogen, methyl, propan-2-yl, propan-1-yl, butan-1-yl, benzyl, imidazol-4-yl-methyl, hydroxymethyl, 1-hydroxyethyl, 2-carboxyethyl, 4-aminobutan-1-yl, 3-aminopropan-1-yl, 2-aminoethyl, aminomethyl or 3-guanidinopropan-1-yl
or
R³ is attached to R¹ and the two together with the atoms to which they are attached form a pyrrolidine or piperidine ring,
R⁴ is hydrogen,
L² is methylene, 1,2-ethylene, 1,3-propylene or ethene-1,2-diyl in which 1,2-ethylene and 1,3-propylene may each be substituted by amino,
   and
   R^{B} is hydrogen or a group of the formula

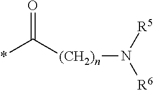

in which
# means the point of linkage to the N atom,
n is the number 1, 2 or 3
and
R⁵ and R⁶ independently of one another are hydrogen or methyl, and the salts, solvates and solvates of the salts thereof.

Particular preference is given to compounds of the formula (I) in which

R^{A} is a group of the formula

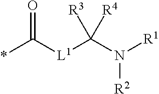

in which
* means the point of linkage to the O atom,
L¹ is a bond,
R¹ and R² are independently of one another hydrogen or methyl,
R³ is hydrogen, methyl, propan-2-yl, propan-1-yl, butan-1-yl, imidazol-4-ylmethyl, hydroxymethyl, 4-aminobutan-1-yl, 3-aminopropan-1-yl, 2-aminoethyl, amino-methyl or 3-guanidinopropan-1-yl and
R⁴ is hydrogen,
   and
   R^{B} is hydrogen,
   and the salts, solvates and solvates of the salts thereof.

The invention further relates to a process for preparing the compounds according to the invention of the formula (I) in which R^{B} is hydrogen, characterized in that

[A] the compound (A)

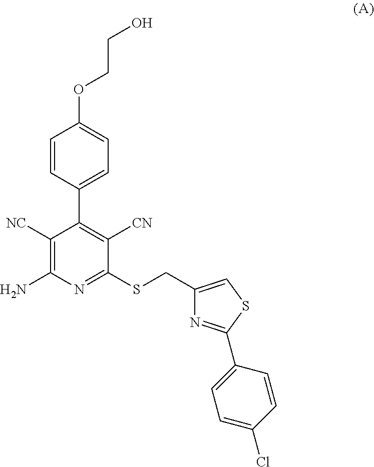

is reacted in an inert solvent in the presence of a base with phosphoryl chloride and subsequently, by heating with water, converted into the compound of the formula (I-A)

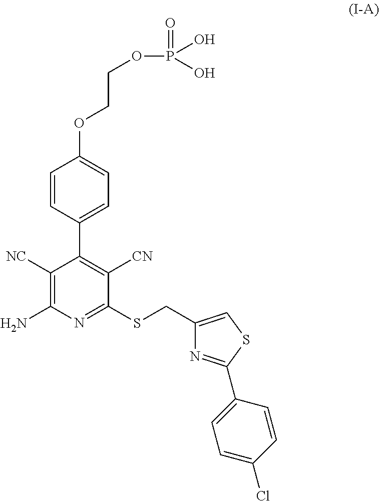

or
[B] the compound (A) is coupled in an inert solvent with a compound of the formula (II)

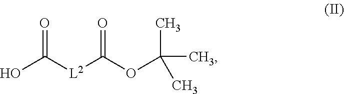

in which L² has the meanings indicated above,
with activation of the carboxyl group in (II) to give a compound of the formula (III)

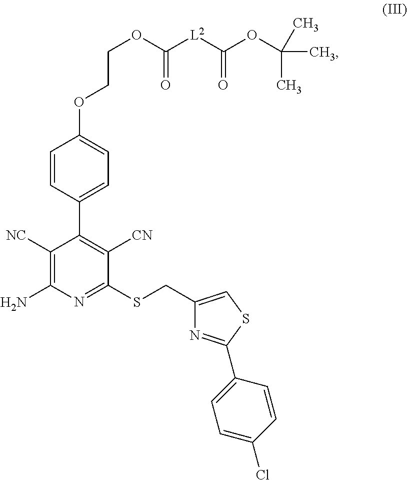

in which L² has the meaning indicated above,
and the tert-butyl ester grouping is then cleaved off with the aid of an acid to give a compound of the formula (I-B)

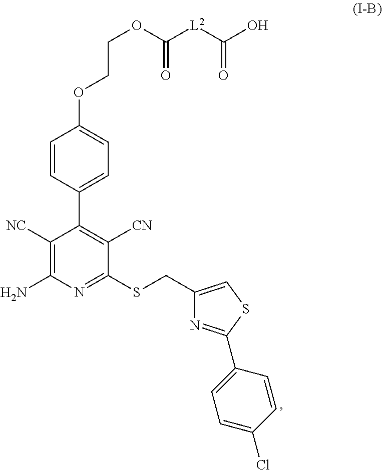

in which L² has the meaning indicated above,
   or
   [C] the compound (A) is coupled in an inert solvent with a compound of the formula (IV)

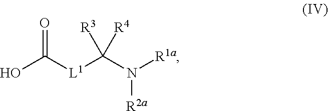

in which L¹, R³ and R⁴ have the meanings indicated above
and
R^{1a} and R^{2a} are identical or different and have the meanings indicated above of R¹ and R², respectively, or are temporary amino protective groups,
with activation of the carboxyl group in (IV) to give a compound of the formula (V)

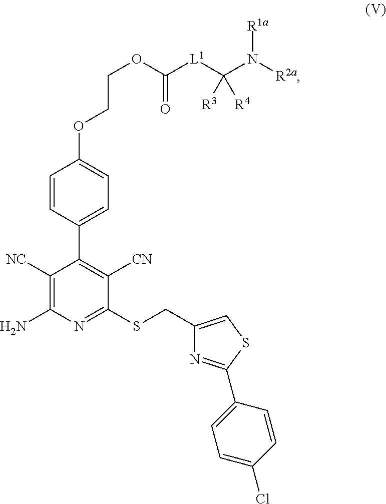

in which L¹, R^{1a}, R^{2a}, R³ and R⁴ have the meanings indicated above,
and any protective groups present are then removed giving a compound of the formula (I-C)

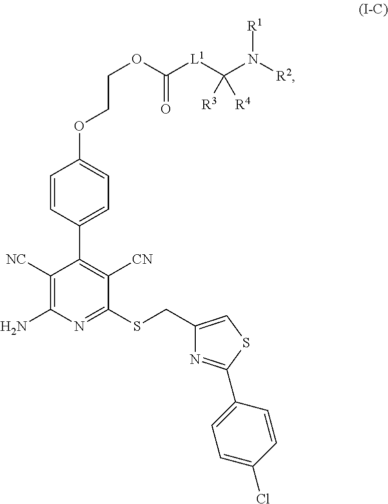

in which L¹, R¹, R², R³ and R⁴ have the meanings indicated above,
   and the resulting compounds of the formulae (I-A), (I-B) and (I-C), respectively, are converted where appropriate with the appropriate (i) solvents and/or (ii) acids or bases into the solvates, salts and/or solvates of the salts thereof.

The compounds of the formulae (I-A), (I-B) and (I-C) may also result directly in the form of salts in the preparation by the processes described above. These salts can be converted where appropriate by treatment with a base or acid in an inert solvent, by chromatographic methods or by ion exchanger resins, into the respective free bases or acids. Further salts of the compounds according to the invention can also be prepared where appropriate by exchange of counterions by means of ion exchange chromatography, for example with Amberlite® resins.

Functional groups which are present where appropriate in the radicals R¹, R², R³ and/or L² of the compounds of the formulae (II) and (IV)—such as, in particular, amino, guanidino, hydroxyl, mercapto and carboxyl groups—may, if expedient or necessary, also be in temporarily protected form in the reaction sequences described above. The introduction and removal of such protective groups takes place in this connection by conventional methods known from peptide chemistry [see, for example, T. W. Greene and P. G. M. Wuts, Protective Groups in Organic Synthesis, Wiley, New York, 1999; M. Bodanszky and A. Bodanszky, The Practice of Peptide Synthesis, Springer-Verlag, Berlin, 1984].

The amino and guanidino protective group which is preferably used is tert-butoxycarbonyl (Boc) or benzyloxycarbonyl (Z). The protective group preferably employed for a hydroxyl or carboxyl function is preferably tert-butyl or benzyl. Elimination of these protective groups is carried out by conventional methods, preferably by reaction with a strong acid such as hydrogen chloride, hydrogen bromide or trifluoroacetic acid in an inert solvent such as dioxane, dichloromethane or acetic acid; the elimination can where appropriate also take place without an additional inert solvent. In the case of benzyl and benzyloxycarbonyl as protective group, these can also be removed by hydrogenolysis in the presence of a palladium catalyst. Elimination of the protective groups mentioned may where appropriate be carried out simultaneously in a one-pot reaction or in separate reaction steps.

The reaction of compound (A) with phosphoryl chloride is preferably carried out in dichloromethane, tetrahydrofuran or dioxane as inert solvent in a temperature range of from −20° C. to +30° C. Suitable bases are in particular tertiary amine bases such as triethylamine. The subsequent hydrolysis to the dihydrogenphosphate (I-A) is carried out by heating the reaction mixture with water at temperatures of from +50° C. to +100° C.

Inert solvents for the coupling reaction (ester formation) in process steps (A)+(II)→(III) and (A)+(IV)→(V) are, for example, ethers, such as diethyl ether, tert-butyl methyl ether, dioxane, tetrahydrofuran, glycol dimethyl ether or diethylene glycol dimethyl ether, hydrocarbons such as benzene, toluene, xylene, hexane, cyclohexane or mineral oil fractions, halogenated hydrocarbons, such as dichloromethane, trichloromethane, carbon tetrachloride, 1,2-dichloroethane, trichloroethylene or chlorobenzene, or other solvents, such as acetone, ethyl acetate, pyridine, dimethyl sulfoxide, dimethylformamide, N,N′-dimethylpropyleneurea (DMPU), N-methylpyrrolidone (NMP) or acetonitrile. It is also possible to use mixtures of the solvents mentioned, Preference is given to dichloromethane, dimethylformamide or mixtures of these two solvents.

Suitable for activating the carboxyl group in compound (II) and (IV) in these coupling reactions are, for example, carbodiimides, such as N,N′-diethyl-, N,N′-dipropyl-, N,N′-diisopropyl-, N,N′ dicyclohexylcarbodiimide (DCC) or N-(3-dimethylaminoisopropyl)-N′-ethylcarbodiimide hydrochloride (EDC), phosgene derivatives, such as N,N′-carbonyldiimidazole (CD), 1,2-oxazolium compounds, such as 2-ethyl-5-phenyl-1,2-oxazolium-3-sulfate or 2-tert-butyl-5-methyl-isoxazolium perchlorate, acylamino compounds, such as 2-ethoxy-1-ethoxycarbonyl-1,2-di-hydroquinoline, or isobutyl chloroformate, propanephosphonic anhydride, diethyl cyano-phosphonate, bis-(2-oxo-3-oxazolidinyl)phosphoryl chloride, benzotriazol-1-yloxytris(dimethyl-amino)phosphonium hexafluorophosphate, benzotriazol-1-yloxytris(pyrrolidino)phosphonium hexafluorophosphate (PyBOP), O-(benzotriazol-1-yl)-N,N,N′,N′-tetramethyluronium tetrafluoro-borate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries such as 1-hydroxybenzotriazole (HOBt) or N-hydroxysuccinimide (HOSu), and also, as bases, alkali metal carbonates, for example sodium carbonate or potassium carbonate, or organic bases, such as triethylamine, N-methyl-morpholine, N-methylpiperidine, N,N-diisopropylethylamine or 4-N,N-dimethylaminopyridine. Preference is given to using N-(3-dimethylaminoisopropyl)-N′-ethylcarbodiimide hydrochloride (EDC) in combination with 4-N,N-dimethylaminopyridine.

The reactions (A)+(II)→(III) and (A)+(IV)→(V) are generally carried out in a temperature range of from 0° C. to +50° C., preferably from +20° C. to +40° C. The reactions can be carried out at atmospheric, at elevated or at reduced pressure (for example from 0.5 to 5 bar). In general, the reactions are carried out at atmospheric pressure.

The compounds of the formula (I) according to the invention in which R^{B} is not hydrogen can be prepared analogously to the reaction sequence (A)+(IV)→(V)→(I-C) by coupling one of the above-described compounds of the formulae (I-A), (III), (I-B), (V) and (I-C) in an inert solvent with a compound of the formula (VI)

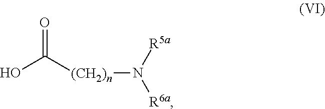

in which n has the meaning indicated above
and
R^{5a} and R^{6a} are identical or different and have the meanings indicated above of R⁵ and R⁶, respectively, or are temporary amino protective groups,
   with activation of the carboxyl group in (VI), followed, if appropriate, by the removal of any protective groups present.

For the coupling reaction with compound (VI) (amide formation), the reaction parameters such as solvents and activating agents described above for the reaction (A)+(IV)→(V) are applied in an analogous manner. The reaction with compound (VI) is preferably carried out in a temperature range of from +20° C. to +60° C.

If R^{A} and R^{B} in formula (I) denote identical groupings, the corresponding compounds according to the invention can also be prepared by reacting compound (A) in a one-pot reaction with an excess of compound (VI).

The compounds of the formulae (II), (IV) and (VI) are commercially available, known from the literature or can be prepared by methods customary in the literature. Thus, for example, compounds of the formula (IV) in which L¹ is —CH₂— or —CH₂CH₂— can be obtained by known methods for extending the chain of carboxylic acids, such as, for example, the Arndt-Eistert reaction [Eistert et al., Ber. Dtsch. Chem. Ges. 60, 1364-1370 (1927); Ye et al., Chem. Rev. 94, 1091-1160 (1994); Cesar et al., Tetrahedron Lett. 42, 7099-7102 (2001)], the derivatization with Meldrum's acid [cf. Smrcina, Tetrahedron 53, 12 867-12 874 (1997)] or the reaction with N-hydroxy-2-thiopyridone [cf. Barton et al., Tetrahedron Lett. 32, 3309-3312 (1991)], in each case starting with compounds of the formula (IV) in which L¹ is a bond.

Preparation of compound (A), 2-amino-6-({[2-(4-chlorophenyl)-1,3-thiazol-4-yl]methyl}thio)-4-[4-(2-hydroxyethoxy)phenyl]pyridine-3,5-dicarbonitrile, is described in WO 03/053441 as example 6.

The preparation of the compounds according to the invention can be illustrated by the following synthesis schemes:

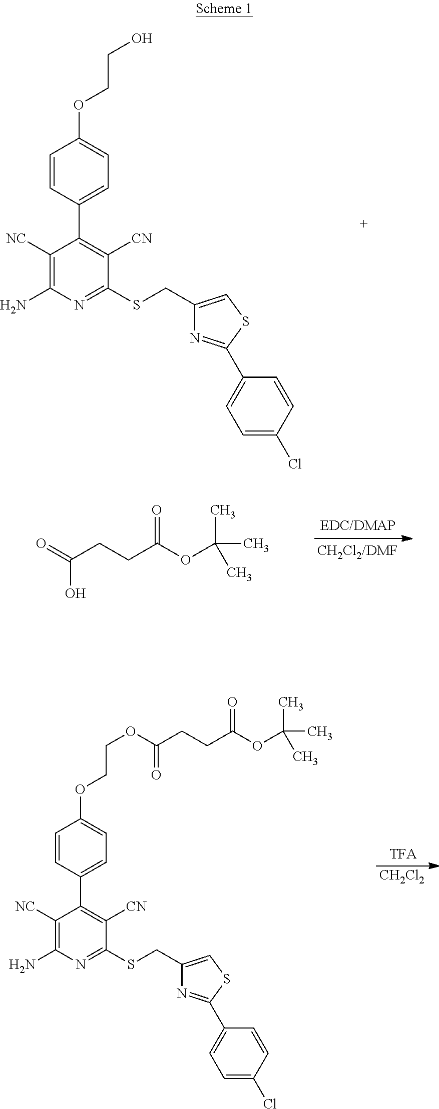

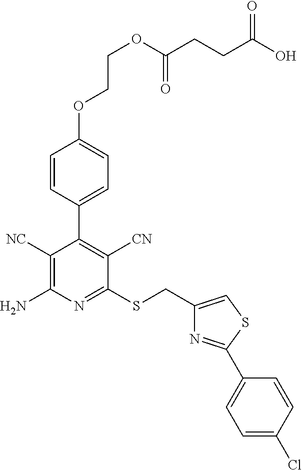

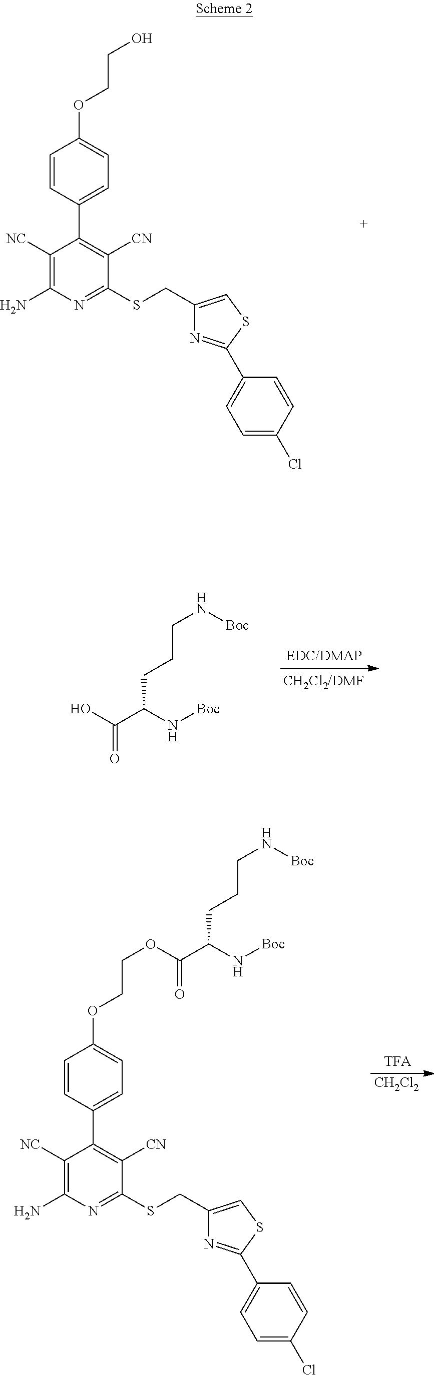

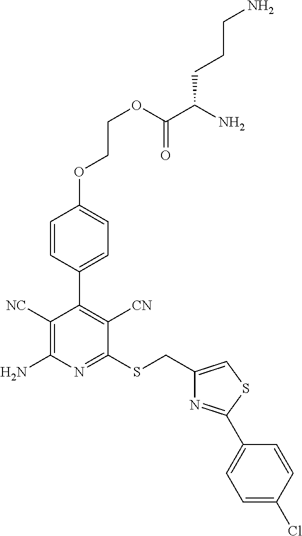

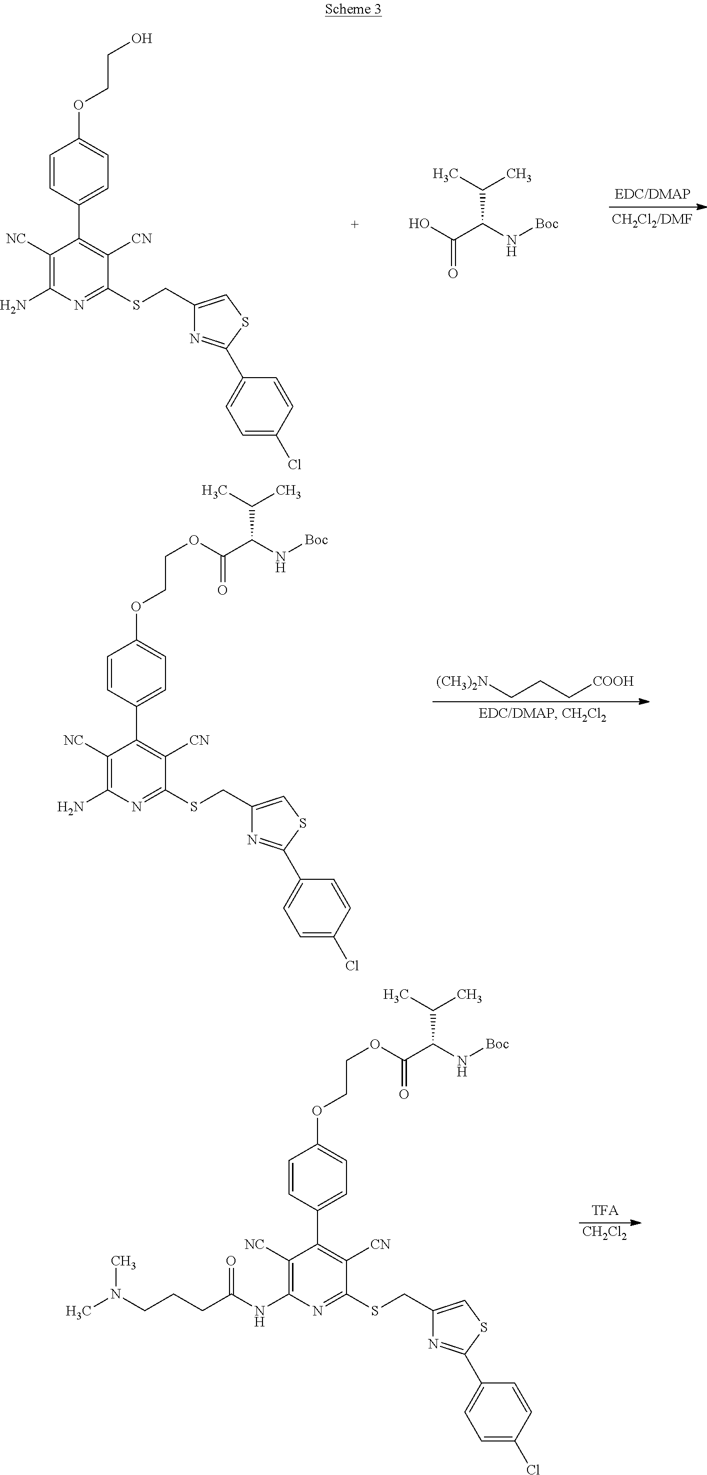

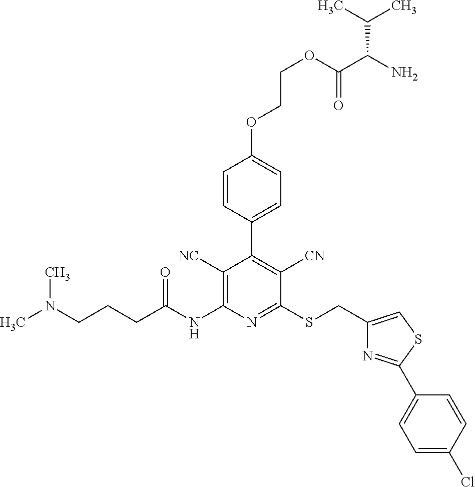

The compounds according to the invention and their salts represent useful prodrugs of the active ingredient compound (A). On the one hand, they show good stability at various pH values and, on the other hand, they show efficient conversion into the active ingredient compound (A) at a physiological pH and in particular in vivo. The compounds according to the invention moreover have improved solubilities in aqueous or other physiologically tolerated media, making them suitable for therapeutic use, in particular on intravenous administration. In addition, the bioavailability from suspension after oral administration is improved by comparison with the parent substance (A).

The compounds of the formula (I) are suitable alone or in combination with one or more other active ingredients for the prophylaxis and/or treatment of various disorders, for example and in particular disorders of the cardiovascular system (cardiovascular disorders), for cardio protection following lesions of the heart, and of metabolic disorders.

Disorders of the cardiovascular system, or cardiovascular disorders, mean in the context of the present invention for example the following disorders: hypertension (high blood pressure), peripheral and cardiac vascular disorders, coronary heart disease, coronary restenosis such as, for example, restenosis following balloon dilatation of peripheral blood vessels, myocardial infarction, acute coronary syndrome, acute coronary syndrome with ST elevation, acute coronary syndrome without ST elevation, stable and unstable angina pectoris, myocardial insufficiency, princemetal angina, persistent ischemic dysfunction (“hibernating myocardium”), temporary postischemic dysfunction (“stunned myocardium”), heart failure, tachycardia, atrial tachycardia, arrhythmias, atrial and ventricular fibrillation, persistent atrial fibrillation, permanent atrial fibrillation, atrial fibrillation with normal left ventricular function, atrial fibrillation with impaired left ventricular function, Wolff-Parkinson-White syndrome, disturbances of peripheral blood flow, elevated levels of fibrinogen and of low density LDL, and elevated concentrations of plasminogen activator inhibitor 1 (PAI-1), especially hypertension, coronary heart disease, acute coronary syndrome, angina pectoris, heart failure, myocardial infarction and atrial fibrillation.

In the context of the present invention, the term heart failure includes both acute and chronic manifestations of heart failure, as well as more specific or related types of disease, such as acute decompensated heart failure, right heart failure, left heart failure, global failure, ischemic cardiomyopathy, dilated cardiomyopathy, congenital heart defects, heart valve defects, heart failure associated with heart valve defects, mitral stenosis, mitral insufficiency, aortic stenosis, aortic insufficiency, tricuspid stenosis, tricuspid insufficiency, pulmonary stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, and diastolic and systolic heart failure.

The compounds according to the invention are further also suitable in particular for reducing the area of myocardium affected by an infarction, and for the prophylaxis of secondary infarctions.

The compounds according to the invention are furthermore suitable in particular for the prophylaxis and/or treatment of thromboembolic disorders, reperfusion damage following ischemia, micro- and macrovascular lesions (vasculitis), arterial and venous thromboses, edemas, ischemias such as myocardial infarction, stroke and transient ischemic attacks, for cardio protection in connection with coronary artery bypass operations (CABG), primary percutaneous transluminal coronary angioplasties (PTCAs), PTCAs after thrombolysis, rescue PTCA, heart transplants and open-heart operations, and for organ protection in connection with transplants, bypass operations, catheter investigations and other surgical procedures.

Further indication areas for which the compounds according to the invention can be used are for example the prophylaxis and/or treatment of disorders of the urogenital region, such as, for example, acute renal failure, unstable bladder, urogenital incontinence, erectile dysfunction and female sexual dysfunction, but also the prophylaxis and/or treatment of inflammatory disorders such as, for example, inflammatory dermatoses and arthritis, especially rheumatoid arthritis, of disorders of the central nervous system and neurodegenerative impairments (post-stroke conditions, Alzheimer's disease, Parkinson's disease, dementia, Huntington's chorea, epilepsy, depression, multiple sclerosis), of painful conditions and migraine, hepatic fibrosis and cirrhosis of the liver, of cancers and of nausea and vomiting in connection with cancer therapies, and for wound healing.

A further indication area is for example the prophylaxis and/or treatment of respiratory disorders such as, for example, asthma, chronic obstructive respiratory disorders (COPD, chronic bronchitis), pulmonary emphysema, bronchiectasies, cystic fibrosis (mucoviscidosis) and pulmonary hypertension, especially pulmonary aterial hypertension.

Finally, the compounds according to the invention are also suitable for the prophylaxis and/or treatment of metabolic disorders such as, for example, diabetes, especially diabetes mellitus, gestational diabetes, insulin-dependent diabetes and non-insulin-dependent diabetes, diabetic sequelae such as, for example, retinopathy, nephropathy and neuropathy, metabolic disorders such as, for example, metabolic syndrome, hyperglycemia, hyperinsulinemia, insulin resistance, glucose intolerance and obesity (adiposity), and arteriosclerosis and dyslipidemias (hypercholesterolemia, hypertriglyceridemia, elevated concentrations of post-prandial plasma triglycerides, hypoalphalipoproteinemia, combined hyperlipidemias), especially, of diabetes, metabolic syndrome and dyslipidemias.

The present invention further relates to the use of the compounds according to the invention for the treatment and/or prophylaxis of disorders, especially of the aforementioned disorders.

The present invention further relates to the use of the compounds according to the invention for the manufacture of a medicament for the treatment and/or prophylaxis of disorders, especially of the aforementioned disorders.

The present invention further relates to a method for the treatment and/or prophylaxis of disorders, especially of the aforementioned disorders, by using an effective amount of at least one of the compounds according to the invention.

The compounds according to the invention can be employed alone or, if required, in combination with other active ingredients. The present invention further relates to medicaments comprising at least one of the compounds according to the invention and one or more further active ingredients, in particular for the treatment and/or prophylaxis of the aforementioned disorders.

Suitable combination active ingredients which may be mentioned by way of example and preferably are: lipid metabolism-altering active ingredients, antidiabetics, blood pressure-reducing agents, agents which promote blood flow and/or have antithrombotic effects, antiarrhythmics, antioxidants, chemokine receptor antagonists, p38 kinase inhibitors, NPY agonists, orexin agonists, anorectic agents, PAF-AH inhibitors, anti-inflammatory agents (COX inhibitors, LTB₄ receptor antagonists), and analgesics such as, for example, aspirin.

The present invention relates in particular to combinations of at least one of the compounds according to the invention with at least one lipid metabolism-altering active ingredient, antidiabetic, blood pressure-reducing active ingredient, antiarrhythmic and/or agent having antithrombotic effects.

The compounds according to the invention can preferably be combined with one or more
lipid metabolism-altering active ingredients, by way of example and preferably from the group of HMG-CoA reductase inhibitors, inhibitors of HMG-CoA reductase expression, squalene synthesis inhibitors, ACAT inhibitors, LDL receptor inducers, cholesterol absorption inhibitors, polymeric bile acid adsorbents, bile acid reabsorption inhibitors, MTP inhibitors, lipase inhibitors, LPL activators, fibrates, niacin, CETP inhibitors, PPAR-α, PPAR-γ and/or PPAR-δ agonists, RXR modulators, FXR modulators, LXR modulators, thyroid hormones and/or thyroid mimetics, ATP-citrate lyase inhibitors, Lp(a) antagonists, cannabinoid receptor 1 antagonists, leptin receptor agonists, bombesin receptor agonists, histamine receptor agonists, and of antioxidants/radical scavengers;
antidiabetics which are mentioned in the Rote Liste 2004/II, Chapter 12, and, by way of example and preferably, those from the group of sulfonylureas, biguanides, meglitinide derivatives, glucosidase inhibitors, inhibitors of dipeptidyl-peptidase IV (DPP-IV inhibitors), oxadiazolidinones, thiazolidinediones, GLP 1 receptor agonists, glucagon antagonists, insulin sensitizers, CCK 1 receptor agonists, leptin receptor agonists, inhibitors of hepatic enzymes involved in the stimulation of gluconeogenesis and/or glycogenolysis, modulators of glucose uptake, and of potassium channel openers such as, for example, those disclosed in WO 97/26265 and WO 99/03861;
blood pressure-reducing active ingredients, by way of example and preferably from the group of calcium antagonists, angiotensin AII antagonists, ACE inhibitors, renin inhibitors, beta-adrenoceptor antagonists, alpha-adrenoceptor antagonists, diuretics, aldosterone antagonists, mineralocorticoid receptor antagonists, ECE inhibitors, and of vasopeptidase inhibitors;
agents having antithrombotic effects, by way of example and preferably from the group of platelet aggregation inhibitors or of anticoagulants;
antiarrhythmics, especially those for the treatment of supraventricular arrhythmias and tachycardias;
substances for the prophylaxis and treatment of ischemic and reperfusion damage;
vasopressin receptor antagonists;
organic nitrates and NO donors;
compounds with positive inotropic activity;
compounds which inhibit the degradation of cyclic guanosine monophosphate (cGMP) and/or cyclic adenosine monophosphate (cAMP), such as, for example, inhibitors of phosphodiesterases (PDE) 1, 2, 3, 4 and/or 5, especially PDE 5 inhibitors such as sildenafil, vardenafil and tadalafil, and PDE 3 inhibitors such as milrinone;
natriuretic peptides such as, for example, atrial natriuretic peptide (ANP, anaritide), B-type natriuretic peptide or brain natriuretic peptide (BNP, nesiritide), C-type natriuretic peptide (CNP) and urodilatin;
agonists of the prostacyclin receptor (IP receptor), such as, for example iloprost, beraprost and cicaprost;
calcium sensitizers such as by way of example and preferably levosimendan;
potassium supplements;
NO and heme-independent activators of guanylate cyclase, such as in particular the compounds described in WO 01/19355, WO 01/19776, WO 01/19778, WO 01/19780, WO 02/070462 and WO 02/070510;
NO-independent but heme-dependent stimulators of guanylate cyclase, such as in particular the compounds described in WO 00/06568, WO 00/06569, WO 02/42301 and WO 03/095451;
Inhibitors of human neutrophile elastase (HNE), such as, for example, sivelestat and DX-890 (reltran);
compounds which inhibit the signal transduction cascade, such as, for example, tyrosine kinase inhibitors, especially sorafenib, imatinib, gefitinib and erlotinib;
compounds which influence the energy metabolism of the heart, such as, for example, etomoxir, dichloroacetate, ranolazine and trimetazidine;
analgesics; and/or
substances for the prophylaxis and treatment of nausea and vomiting

Lipid metabolism-altering active ingredients preferably mean compounds from the group of HMG-CoA reductase inhibitors, squalene synthesis inhibitors, ACAT inhibitors, cholesterol absorption inhibitors, MTP inhibitors, lipase inhibitors, thyroid hormones and/or thyroid mimetics, niacin receptor agonists, CETP inhibitors, PPAR-α agonists, PPAR-γ agonists, PPAR-δ agonists, polymeric bile acid adsorbents, bile acid reabsorption inhibitors, antioxidants/radical scavengers, and cannabinoid receptor 1 antagonists.

In a preferred embodiment of the invention, the compounds according to the invention are administered in combination with an HMG-CoA reductase inhibitor from the class of statins, such as by way of example and preferably lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor, such as by way of example and preferably BMS-188494 or TAK-475.

In a preferred embodiment of the invention, the compounds according to the invention are administered in combination with an ACAT inhibitor, such as by way of example and preferably avasimibe, melinamide, pactimibe, eflucimibe or SMP-797.

In a preferred embodiment of the invention, the compounds according to the invention are administered in combination with a cholesterol absorption inhibitor, such as by way of example and preferably ezetimibe, tiqueside or pamaqueside.

In a preferred embodiment of the invention, the compounds according to the invention are administered in combination with an MTP inhibitor, such as by way of example and preferably implitapide, BMS-201038, R-103757 or JTT-130.

In a preferred embodiment of the invention, the compounds according to the invention are administered in combination with a lipase inhibitor, such as by way of example and preferably orlistat.

In a preferred embodiment of the invention, the compounds according to the invention are administered in combination with a thyroid hormone and/or thyroid mimetic, such as by way of example and preferably D-thyroxine or 3,5,3′-triiodothyronine (T3).

In a preferred embodiment of the invention, the compounds according to the invention are administered in combination with an agonist of the niacin receptor, such as by way of example and preferably niacin, acipimox, acifran or radecol.

In a preferred embodiment of the invention, the compounds according to the invention are administered in combination with a CETP inhibitor, such as by way of example and preferably torcetrapib, JTT-705, BAY 60-5521, BAY 78-7499 or CETP vaccine (Avant).

In a preferred embodiment of the invention, the compounds according to the invention are administered in combination with a PPAR-γ agonist, such as by way of example and preferably pioglitazone or rosiglitazone.

In a preferred embodiment of the invention, the compounds according to the invention are administered in combination with a PPAR-δ agonist, such as by way of example and preferably GW-501516 or BAY 68-5042.

In a preferred embodiment of the invention, the compounds according to the invention are administered in combination with a polymeric bile acid adsorbent, such as by way of example and preferably cholestyramine, colestipol, colesolvam, CholestaGel or colestimide.

In a preferred embodiment of the invention, the compounds according to the invention are administered in combination with a bile acid reabsorption inhibitor, such as by way of example and preferably ASBT (=IBAT) inhibitors, such as, for example, AZD-7806, S-8921, AK-105, BARI-1741, SC-435 or SC-635.

In a preferred embodiment of the invention, the compounds according to the invention are administered in combination with an antioxidant/radical scavenger, such as by way of example and preferably probucol, AGI-1067, BO-653 or AEOL-10150.

In a preferred embodiment of the invention, the compounds according to the invention are administered in combination with a cannabinoid receptor 1 antagonist, such as by way of example and preferably rimonabant or SR-147778.

Antidiabetics preferably mean insulin and insulin derivatives, and orally active hypoglycemic active ingredients. Insulin and insulin derivatives includes in this connection both insulins of animal, human or biotechnological origin and mixtures thereof. The orally active hypoglycemic active ingredients preferably include sulfonylureas, biguanides, meglitinide derivatives, glucosidase inhibitors, DPP-IV inhibitors and PPAR-γ agonists.

In a preferred embodiment of the invention, the compounds according to the invention are administered in combination with insulin.

In a preferred embodiment of the invention, the compounds according to the invention are administered in combination with a sulfonylurea, such as by way of example and preferably tolbutamide, glibenclamide, glimepiride, glipizide or gliclazide.

In a preferred embodiment of the invention, the compounds according to the invention are administered in combination with a biguanide, such as by way of example and preferably metformin.

In a preferred embodiment of the invention, the compounds according to the invention are administered in combination with a meglitinide derivative, such as by way of example and preferably repaglinide or nateglinide.

In a preferred embodiment of the invention, the compounds according to the invention are administered in combination with a glucosidase inhibitor, such as by way of example and preferably miglitol or acarbose.

In a preferred embodiment of the invention, the compounds according to the invention are administered in combination with a DPP-IV inhibitor, such as by way of example and preferably sitagliptin or vildagliptin.

In a preferred embodiment of the invention, the compounds according to the invention are administered in combination with a PPAR-γ agonist, for example from the class of thiazolidinediones, such as by way of example and preferably pioglitazone or rosiglitazone.

Blood pressure-reducing agents preferably mean compounds from the group of calcium antagonists, angiotensin AII antagonists, ACE inhibitors, renin inhibitors, beta-adrenoceptor antagonists, alpha-adrenoceptor antagonists and diuretics.

In a preferred embodiment of the invention, the compounds according to the invention are administered in combination with a calcium antagonist, such as by way of example and preferably nifedipine, amlodipine, verapamil or diltiazem.

In a preferred embodiment of the invention, the compounds according to the invention are administered in combination with an angiotensin AII antagonist, such as by way of example and preferably losartan, valsartan, candesartan, embusartan, olmesartan or telmisartan.

In a preferred embodiment of the invention, the compounds according to the invention are administered in combination with an ACE inhibitor, such as by way of example and preferably enalapril, captopril, lisinopril, ramipril, delapril, fosinopril, quinopril, perindopril or trandopril.

In a preferred embodiment of the invention, the compounds according to the invention are administered in combination with a renin inhibitor, such as by way of example and preferably aliskiren, SPP-600 or SPP-800.

In a preferred embodiment of the invention, the compounds according to the invention are administered in combination with a beta-adrenoceptor antagonist, such as by way of example and preferably propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an alpha-adrenoceptor antagonist, such as by way of example and preferably prazosin.

In a preferred embodiment of the invention, the compounds according to the invention are administered in combination with a diuretic, such as by way of example and preferably furosemide, bumetanide, torsemide, bendroflumethiazide, chlorothiazide, hydrochlorothiazide, hydroflumethiazide, methyclothiazide, polythiazide, trichlormethiazide, chlorthalidone, indapamide, metolazone, quinethazone, acetazolamide, dichlorophenamide, methazolamide, glycerol, isosorbide, mannitol, amiloride or triamterene.

In a preferred embodiment of the invention, the compounds according to the invention are administered in combination with an aldosterone or mineralocorticoid receptor antagonist, such as by way of example and preferably spironolactone or eplerenone.

In a preferred embodiment of the invention, the compounds according to the invention are administered in combination with a vasopressin receptor antagonist, such as by way of example and preferably conivaptan, tolvaptan, lixivaptan or SR-121463.

In a preferred embodiment of the invention, the compounds according to the invention are administered in combination with an organic nitrate or NO donor, such as by way of example and preferably sodium nitroprusside, glycerol nitrate, isosorbide mononitrate, isosorbide dinitrate, molsidomine or SIN-1, or in combination with inhaled NO.

In a preferred embodiment of the invention, the compounds according to the invention are administered in combination with a compound having positive inotropic activity, such as by way of example and preferably cardiac glycosides (digoxin) and beta-adrenergic and dopaminergic agonists such as isoproterenol, adrenaline, noradrenaline, dopamine or dobutamine.

In a preferred embodiment of the invention, the compounds according to the invention are administered in combination with antisympathotonics such as reserpine, clonidine or alpha-methyldopa, or in combination with potassium channel agonists such as minoxidil, diazoxide, dihydralazine or hydralazine.

Agents having an antithrombotic effect preferably mean compounds from the group of platelet aggregation inhibitors or of anticoagulants.

In a preferred embodiment of the invention, the compounds according to the invention are administered in combination with a platelet aggregation inhibitor, such as by way of example and preferably aspirin, clopidogrel, ticlopidine or dipyridamole.

In a preferred embodiment of the invention, the compounds according to the invention are administered in combination with a thrombin inhibitor, such as by way of example and preferably ximelagatran, melagatran, bivalirudin or clexane.

In a preferred embodiment of the invention, the compounds according to the invention are administered in combination with a GPIIb/IIIa antagonist, such as by way of example and preferably tirofiban or abciximab.

In a preferred embodiment of the invention, the compounds according to the invention are administered in combination with a factor Xa inhibitor, such as by way of example and preferably rivaroxaban (BAY 59-7939), DU-176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as by way of example and preferably coumarin.

Antiarrhythmics preferably means substances from the group of class Ia antiarrhythmics (e.g. quinidine), of class Ic antiarrhythmics (e.g. flecamide, propafenone), of class II antiarrhythmics (e.g. metoprolol, atenolol, sotalol, oxprenolol and other beta-receptor blockers), of class III antiarrhythmics (e.g. sotalol, amiodarone) and of class IV antiarrhythmics (e.g. digoxin, and verapamil, diltiazem and other calcium antagonists).

Particular preference is given in the context of the present invention to combinations comprising at least one of the compounds according to the invention and one or more further active ingredients selected from the group consisting of HMG-CoA reductase inhibitors (statins), diuretics, beta-adrenoceptor antagonists, alpha-adrenoceptor antagonists, organic nitrates and NO donors, calcium antagonists, ACE inhibitors, angiotensin AII antagonists, aldosterone and mineralocorticoid receptor antagonists, vasopressin receptor antagonists, platelet aggregation inhibitors, anticoagulants and antiarrhythmics, and to the use thereof for the treatment and/or prophylaxis of the aforementioned disorders.

The present invention further relates to medicaments which comprise at least one compound according to the invention, normally together with one or more inert, non-toxic, pharmaceutically suitable excipients, and to the use thereof for the aforementioned purposes.

The compounds according to the invention can act systemically and/or locally. For this purpose, they can be administered in a suitable way such as, for example, by the oral, parenteral, pulmonary, nasal, sublingual, lingual, buccal, rectal, dermal, transdermal, conjunctival or otic route or as an implant or stent. The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions or sprays, tablets for lingual, sublingual or buccal administration, films/wafers or capsules, suppositories, preparations for the eyes or ears, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

Oral or parenteral administration is preferred, especially oral and intravenous administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxy-sorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and masking flavors and/or odors.

It has generally proved advantageous to administer on parenteral administration amounts of about 0.001 to 1 mg/kg, preferably about 0.01 to 0.5 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. Examples

### Abbreviations and Acronyms

Boc tert-butoxycarbonyl
DMAP 4-N,N-dimethylaminopyridine
DMF N,N-dimethylformamide
DMSO dimethyl sulfoxide
EDC 1-(3-dimethylaminopropyl)-3-ethylcarbodiimide hydrochloride
ESI electrospray ionization (in MS)
h hour(s)
HPLC high pressure, high performance liquid chromatography
LC-MS coupled liquid chromatography-mass spectrometry
min minute(s)
MS mass spectrometry
NMR nuclear magnetic resonance spectrometry
p para
quant. quantitative (for yield)
RT room temperature
Rₜ retention time (in HPLC)
tert. tertiary
TFA trifluoroacetic acid
THF tetrahydrofuran
UV ultraviolet spectrometry
v/v volume to volume ratio (of a solution)
Z benzyloxycarbonyl

### LC-MS and HPLC Methods:

### Method 1 (LC-MS):

MS instrument type: Micromass ZQ; HPLC instrument type: Waters Alliance 2795; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 2 (LC-MS):

Instrument: Micromass Quattro LCZ with HPLC Agilent Series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 3 (LC-MS):

MS instrument type: Micromass ZQ; HPLC instrument type: HP 1100 Series; UV DAD; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 4 (LC-MS):

MS instrument type: Micromass ZQ; HPLC instrument type: HP 1100 Series; UV DAD; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 5 (Preparative HPLC):

HPLC instrument type: Abimed/Gilson Pump 305/306; Manometric Module 806; UV Knauer Variable Wavelength Monitor; column: Gromsil C18, 10 nm, 250 mm×30 mm; eluent A: 1 l water+0.5 ml 99% trifluoroacetic acid, eluent B: 1 l acetonitrile; gradient: 0.0 min 2% B→10 min 2% B→50 min 90% B; flow rate: 20 ml/min; volume: 628 ml A and 372 ml B.

### Method 6a (Preparative HPLC):

Column: VP 250/21 Nukleodur 100-5 C18 ec, Macherey & Nagel Nr. 762002; eluent A: water/0.01% trifluoroacetic acid, eluent B: acetonitrile/0.01% trifluoroacetic acid; gradient: 0 min 0% B→20 min 20% B→40 min 20% B→60 min 30% B→80 min 30% B→90 min 100% B→132 min 100% B; flow rate: 5 ml/min; temperature: RT; UV detection: 210 nm.

### Method 6b (Preparative HPLC):

Column: VP 250/21 Nukleodur 100-5 C18 ec, Macherey & Nagel Nr. 762002; eluent A: 1 liter water/1 ml 99% trifluoroacetic acid, eluent B: 1 liter acetonitrile/1 ml 99% trifluoroacetic acid; gradient: 0 min 30% B→20 min 50% B→40 min 80% B→60 min 100% B; flow rate: 5 ml/min; temperature: RT; UV detection: 210 nm.

### Method 7 (Analytical HPLC):

Column: XTerra 3.9×150 WAT 186000478; eluent A: 10 ml 70% perchloric acid in 2.5 liters water, eluent B: acetonitrile; gradient: 0.0 min 20% B→1 min 20% B→4 min 90% B→9 min 90% B; temperature: RT; flow rate: 1 ml/min.

### Method 8 (LC-MS):

Instrument: Micromass Quattro LCZ with HPLC Agilent Series 1100; column: Phenomenex Onyx Monolithic C18, 100 mm×3 mm; eluent A: 1 l water+0.5 ml 50% formic acid, eluent B: 1 l acetonitrile+0.5 ml 50% formic acid; gradient: 0.0 min 90% A→2 min 65% A→4.5 min 5% A→6 min 5% A; flow rate: 2 ml/min; oven: 40° C.; UV detection: 208-400 nm.

### Method 9 (LC-MS):

Instrument: Micromass Platform LCZ with HPLC Agilent Series 1100; column: Thermo Hypersil GOLD 3μ, 20 mm×4 mm; eluent A: 1 l water+0.5 ml 50% formic acid, eluent B: 1 l acetonitrile+0.5 ml 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 10 (LC-MS):

MS instrument type: Micromass ZQ; HPLC instrument type: HP 1100 Series; UV DAD; column: Phenomenex Gemini 3μ 30 mm×3.00 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 11 (LC-MS):

MS instrument type: Micromass ZQ; HPLC instrument type: Waters Alliance 2795; column: Phenomenex Synergi 2.5μ MAX-RP 100A Mercury 20 mm×4 mm; eluent A: 1 l water+0.5 ml 50% formic acid, eluent B: 1 l acetonitrile+0.5 ml 50% formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

### Method 12 (LC-MS):

Instrument: Micromass Quattro LCZ with HPLC Agilent Series 1100; column: Phenomenex Synergi 2.5μ MAX-RP 100A Mercury 20 mm×4 mm; eluent A: 1 l water+0.5 ml 50% formic acid, eluent B: 1 l acetonitrile+0.5 ml 50% formic acid; gradient: 0.0 min 90% A→0.1 min 90% A→3.0 min 5% A→4.0 min 5% A→4.1 min 90% A; flow rate: 2 ml/min; oven: 50° C.; UV detection: 208-400 nm.

### EXEMPLARY EMBODIMENTS

### Example 1

### 2-{4-[2-Amino-6-({[2-(4-chlorophenyl)-1,3-thiazol-4-yl]methyl}thio)-3,5-dicyanopyridin-4-yl]-phenoxy}ethyl dihydrogenphosphate

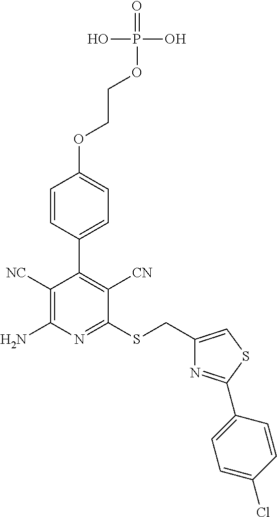

5.35 g (10.29 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile [WO 03/053441, Example 6] are dissolved in 200 ml of THF. 2.15 ml (15.43 mmol) of triethylamine are added, and 1.44 ml (15.43 mmol) of phosphoryl chloride are then added dropwise. The mixture is stirred at room temperature for two hours and then poured into 500 ml of water. The solution is then heated under reflux for one hour, during which time a precipitate is formed. After cooling, this precipitate is filtered off with suction, washed with water and dissolved in DMF. A mixture of water and ethyl acetate is added to this solution, Saturated sodium bicarbonate solution is then added. The phases are separated. The aqueous phase is acidified with 5 N hydrochloric acid. A milky precipitate is formed. The milky suspension is heated to reflux. At room temperature, the precipitate is then filtered off with suction. The residue is twice washed with water, and dried. This gives 5.70 g (92% of theory) of the desired product as a colorless powder.

LC-MS (Method 1): Rₜ=2.44 min; MS (ESIpos): m/z=600 (M+H)⁺

¹H-NMR (400 MHz, DMSO-d₆): δ=4.13-4.28 (m, 4H), 4.64 (br. s, 2H), 7.12 (d, 2H), 7.48 (d, 2H), 7.57 (d, 2H), 7.92 (d, 2H), 7.96 (s, 1H), 8.13 (br. s, 2H).

### Example 2

### 2-{4-[2-Amino-6-({[2-(4-chlorophenyl)-1,3-thiazol-4-yl]methyl}thio)-3,5-dicyanopyridin-4-yl]-phenoxy}ethyl dihydrogenphosphate dipotassium salt

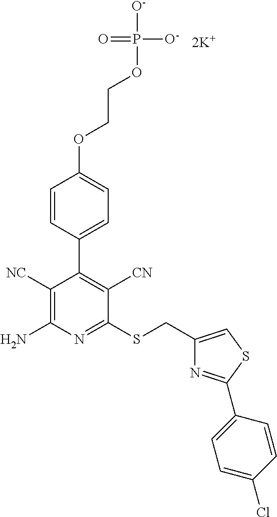

1.00 g (1.57 mmol) of 2-{4-[2-amino-6-({[2-(4-chlorophenyl)-1,3-thiazol-4-yl]methyl}thio)-3,5-di-cyanopyridin-4-yl]phenoxy}ethyl dihydrogenphosphate from Example 1 is dissolved in 15 ml of DMF. 10 ml of water are added, whereupon the solution becomes turbid. 0.46 g (4.71 mmol) of potassium acetate is then added, and the solution becomes clear again. The solution is then concentrated on a rotary evaporator (about 5 ml of solvent are distilled off). A precipitate is formed, which is filtered off, washed three times with ethyl acetate and dried. The precipitate is dissolved in 15 ml of THF and 3 ml of water. 15 ml of toluene are added. The solvents are then removed again on a rotary evaporator. Three times in total, toluene is added to the residue and the solution is in each case concentrated again. The crystals obtained are dried over phosphorus pentoxide under reduced pressure overnight. This gives 0.98 g (92% of theory) of the desired product as a colorless powder.

LC-MS (Method 1): Rₜ=2.39 min; MS (ESIpos): m/z=600 (M+H)⁺

¹H-NMR (400 MHz, DMSO-d₆): δ=3.89-3.98 (m, 2H), 4.12-4.17 (m, 2H), 4.63 (s, 2H), 7.09 (d, 2H), 7.43 (d, 2H), 7.54 (d, 2H), 7.88-7.97 (m, 3H), 8.10 (br. s, 2H).

### Example 3

### 2-{4-[2-Amino-6-({[2-(4-chlorophenyl)-1,3-thiazol-4-yl]methyl}thio)-3,5-dicyanopyridin-4-yl]-phenoxy}ethyl dihydrogenphosphate calcium salt

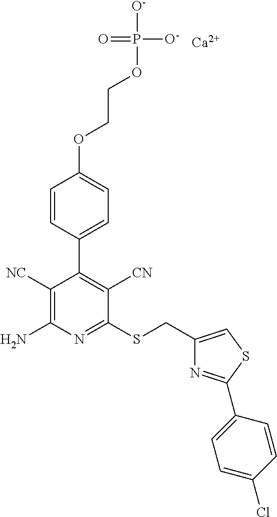

60 mg (0.09 mmol) of 2-{4-[2-amino-6-({[2-(4-chlorophenyl)-1,3-thiazol-4-yl]methyl}thio)-3,5-di-cyanopyridin-4-yl]phenoxy}ethyl dihydrogenphosphate from Example 1 are dissolved in 5 ml of DMF. 5 ml of water and then 27 mg (0.14 mmol) of calcium acetate dihydrate are added. The pH of the solution is 5.6. The solution is then extracted with ethyl acetate, and the organic phase is discarded. On a rotary evaporator, the aqueous phase is concentrated to a volume of about 3 ml. A precipitate is formed, which is filtered off with suction, washed once with water and dried. This gives 29 mg (48% of theory) of the desired product as a colorless powder.

### Example 4

### 2-{4-[2-Amino-6-({[2-(4-chlorophenyl)-1,3-thiazol-4-yl]methyl}thio)-3,5-dicyanopyridin-4-yl]-phenoxy}ethyl dihydrogenphosphate disodium salt

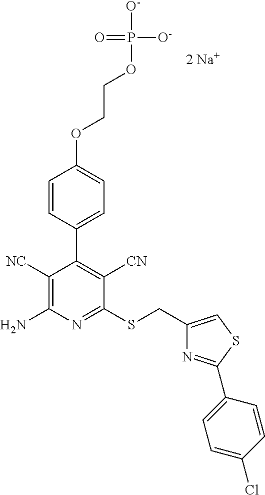

60 mg (0.09 mmol) of 2-{4-[2-amino-6-({[2-(4-chlorophenyl)-1,3-thiazol-4-yl]methyl}thio)-3,5-di-cyanopyridin-4-yl]phenoxy}ethyl dihydrogenphosphate from Example 1 are dissolved in 5 ml of DMF. 5 ml of water and then 23 mg (0.28 mmol) of sodium acetate are added. The pH of the solution is 5.6. The solution is then extracted with ethyl acetate, and the organic phase is discarded. On a rotary evaporator, the aqueous phase is concentrated to a volume of about 3 ml. A precipitate is formed, which is filtered off with suction, washed once with water and dried. This gives 31 mg (51% of theory) of the desired product as a colorless powder.

LC-MS (Method 1): Rₜ=2.30 min; MS (ESIpos): m/z=600 (M+H)⁺

¹H-NMR (400 MHz, DMSO-d₆): δ=3.98-4.23 (m, 4H), 4.54-4.65 (br. s, 2H), 7.12 (d, 2H), 7.38 (d, 2H), 7.53 (d, 2H), 7.83-7.95 (m, 3H), 8.06 (br. s, 2H).

### Example 5

### 2-{4-[2-Amino-6-({[2-(4-chlorophenyl)-1,3-thiazol-4-yl]methyl}thio)-3,5-dicyanopyridin-4-yl]-phenoxy}ethyl glycinate

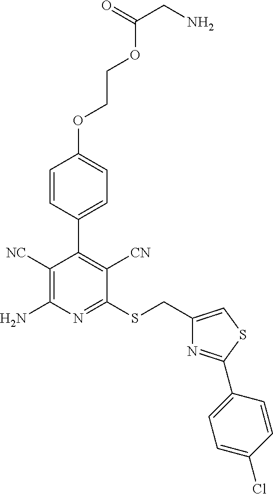

0.250 g (0.481 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.093 g (0.53 mmol) of Boc-glycine, 0.111 g (0.58 mmol) of 1-(3-dimethylaminopropyl)-3-ethylcarbodiimide hydrochloride and 0.006 g (0.048 mmol) of 4-dimethylaminopyridine are combined in 10 ml of dichloromethane and 2.5 ml of DMF and then stirred at room temperature for two hours. A clear solution is formed. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated.

The residue is taken up in 5 ml of dichloromethane, and 1 ml of trifluoroacetic acid is added. The mixture is stirred at room temperature overnight. The reaction mixture is then concentrated to dryness, and the residue is crystallized from a mixture of dichloromethane and diethyl ether. The crystals are filtered off with suction, washed with diethyl ether and dried. The solid is then taken up in ethyl acetate and added to a mixture of semisaturated sodium bicarbonate solution and ethyl acetate. The organic phase is separated off, washed with saturated sodium chloride solution, dried over magnesium sulfate, filtered and concentrated. This gives 220 mg (79% of theory) of the desired product as a colorless powder.

LC-MS (Method 1): Rₜ=1.83 min; MS (ESIpos): m/z=577 (M+H)⁺

¹H-NMR (400 MHz, DMSO-d₆): δ=1.64 (br. s, 2H), 4.26-4.30 (m, 2H), 4.47-4.51 (m, 2H), 4.63 (s, 2H), 7.12 (d, 2H), 7.48 (d, 2H), 7.57 (d, 2H), 7.92 (d, 2H), 7.95 (s, 1H), 8.13 (br. s, 2H).

### Example 6

### 2-{4-[2-Amino-6-({[2-(4-chlorophenyl)-1,3-thiazol-4-yl]methyl}thio)-3,5-dicyanopyridin-4-yl]-phenoxy}ethyl glycinate dihydrochloride

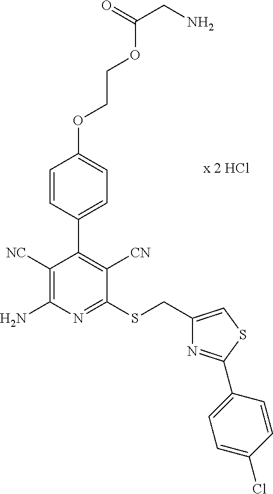

5.57 g (9.64 mmol) of 2-{4-[2-amino-6-({[2-(4-chlorophenyl)-1,3-thiazol-4-yl]methyl}thio)-3,5-di-cyanopyridin-4-yl]phenoxy}ethyl glycinate from Example 5 are partially dissolved in 2-propanol, and 5 ml (20 mmol) of a 4 M solution of hydrogen chloride in dioxane are added. The precipitated crystals are filtered off with suction, washed with dichloromethane and diethyl ether and dried. This gives 5.83 g (93% of theory) of the desired product as colorless crystals.

LC-MS (Method 1): Rₜ=1.83 min; MS (ESIpos): m/z=577 (M+H)⁺

¹H-NMR (400 MHz, DMSO-d₆): δ=3.88 (br. s, 2H), 4.28-4.35 (m, 2H), 4.52-4.58 (m, 2H), 4.63 (s, 2H), 7.12 (d, 2H), 7.48 (d, 2H), 7.58 (d, 2H), 7.92 (d, 2H), 7.95 (s, 1H), 8.15 (br. s, 2H), 8.48 (br. s, 2H).

### Example 7

### 2-{4-[2-Amino-6-({[2-(4-chlorophenyl)-1,3-thiazol-4-yl]methyl}thio)-3,5-dicyanopyridin-4-yl]-phenoxy}ethyl L-norleucinate

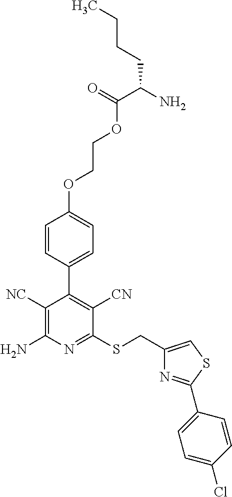

0.100 g (0.19 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.049 g (0.21 mmol) of Boc-L-norleucine, 0.044 g (0.23 mmol) of 1-(3-dimethylaminopropyl)-3-ethylcarbodiimide hydrochloride and 0.002 g (0.02 mmol) of 4-dimethylaminopyridine are combined in 4 ml of dichloromethane and 1 ml of DMF and stirred at room temperature for two hours. A clear solution is formed. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated.

The residue is dissolved in 5 ml of methanol, 1 ml of a 4 N solution of hydrogen chloride in dioxane are added and the solution is stirred at room temperature overnight. The reaction mixture is then added to a mixture of semisaturated sodium bicarbonate solution and ethyl acetate. The organic phase is separated off, washed with saturated sodium chloride solution, dried over magnesium sulfate, filtered and concentrated. The residue is purified by column chromatography on 15 g of silica gel (mobile phase: dichloromethane/ethyl acetate 3:1→dichloromethane/ethyl acetate/methanol 30:10:2). This gives 54 mg (44% of theory) of the desired product as a beige powder.

LC-MS (Method 2): Rₜ=2.10 min; MS (ESIpos): m/z=633 (M+H)⁺

¹H-NMR (400 MHz, DMSO-d₆): δ=0.79 (t, 3H), 1.15-1.31 (m, 4H), 1.38-1.58 (m, 2H), 1.77-1.90 (m, 1H), 4.26-4.32 (m, 2H), 4.33-4.48 (m, 2H), 4.63 (s, 2H), 7.10 (d, 2H), 7.46 (d, 2H), 7.57 (d, 2H), 7.92 (d, 2H), 7.95 (s, 1H), 8.13 (br. s, 2H).

### Example 8

### 2-{4-[2-Amino-6-({[2-(4-chlorophenyl)-1,3-thiazol-4-yl]methyl}thio)-3,5-dicyanopyridin-4-yl]-phenoxy}ethyl L-prolinate bis(trifluoroacetate)

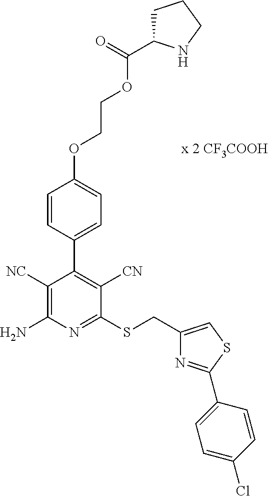

100 mg (0.19 mmol) of 2-amino-6-({[2-(4-chlorophenyl)-1,3-thiazol-4-yl]methyl}thio)-4-[4-(2-hydroxyethoxy)phenyl]-3,5-pyridinedicarbonitrile, 45 mg (0.21 mmol) of Boc-L-proline, 44 mg (0.23 mmol) of 1-(3-dimethylaminopropyl)-3-ethylcarbodiimide hydrochloride and 2 mg (0.02 mmol) of 4-dimethylaminopyridine are combined in 1 ml of DMF and 4 ml of dichloromethane and stirred at room temperature for two hours. A clear solution is formed. The reaction mixture is then added to a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated.

The residue is dissolved in 5 ml of dichloromethane, and 1 ml of trifluoroacetic acid is added. The mixture is stirred at room temperature for 12 hours and then concentrated. The residue is initially crystallized from a mixture of dichloromethane and diethyl ether, and the crystals obtained are washed with diethyl ether. The product is then recrystallized from a mixture of THF and dichloromethane, and the crystals are washed with diethyl ether and dried. This gives 35 mg (22% of theory) of the desired product as colorless crystals.

LC-MS (Method 1): Rₜ=1.91 min; MS (ESIpos): m/z=617 (M+H)⁺

¹H-NMR (400 MHz, DMSO-d₆): δ=1.85-1.95 (m, 2H), 1.97-2.04 (m, 1H), 2.22-2.32 (m, 1H), 3.13-3.26 (m, 2H), 4.30 (s, 2H), 4.43 (t, 1H), 4.50-4.60 (m, 2H), 4.60 (s, 2H), 7.11 (d, 2H), 7.47 (d, 2H), 7.52 (d, 2H), 7.91 (d, 2H), 7.93 (s, 1H), 8.02-8.30 (br. s, 2H), 9.07-9.32 (br. s, 2H).

### Example 9

### 2-{4-[2-Amino-6-({[2-(4-chlorophenyl)-1,3-thiazol-4-yl]methyl}thio)-3,5-dicyanopyridin-4-yl]-phenoxy}ethyl L-prolinate hydrochloride

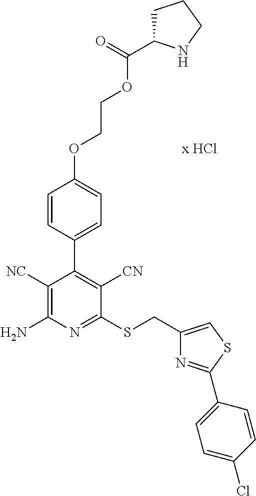

100 mg (0.12 mmol) of 2-{4-[2-amino-6-({[2-(4-chlorophenyl)-1,3-thiazol-4-yl]methyl}thio)-3,5-di-cyanopyridin-4-yl]phenoxy}ethyl L-prolinate bis(trifluoroacetate) from Example 8 are dissolved in 8 ml of DMF, and 0.06 ml of (0.24 mmol) of a 4 M solution of hydrogen chloride in dioxane is added. The precipitated crystals are filtered off with suction and dried under high vacuum. This gives 60 mg (73% of theory) of the desired product as colorless crystals.

LC-MS (Method 3): Rₜ=2.13 min; MS (ESIpos): m/z=617 (M+H)⁺

¹H-NMR (400 MHz, DMSO-d₆): δ=1.85-2.04 (m, 3H), 2.22-2.32 (m, 1H), 3.14-3.30 (m, 2H), 4.32-4.65 (m, 8H), 7.14 (d, 2H), 7.49 (d, 2H), 7.56 (d, 2H), 7.93 (d, 2H), 7.98 (s, 1H), 8.15 (br. s, 2H), 8.93 (br. s, 1H), 9.78 (br. s, 1H).

### Example 10

### 2-{4-[2-Amino-6-({[2-(4-chlorophenyl)-1,3-thiazol-4-yl]methyl}thio)-3,5-dicyanopyridin-4-yl]-phenoxy}ethyl L-prolinate p-toluenesulfonic acid salt

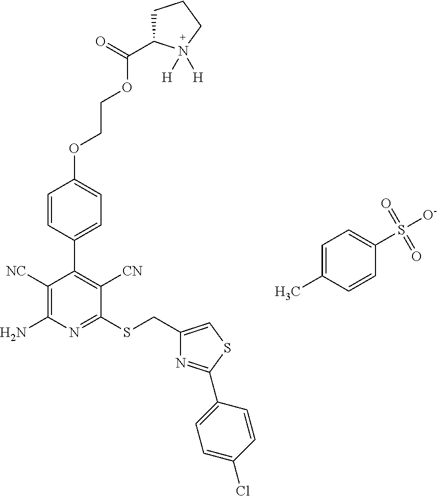

60 mg (0.07 mmol) of 2-{4-[2-amino-6-({[2-(4-chlorophenyl)-1,3-thiazol-4-yl]methyl}thio)-3,5-di-cyanopyridin-4-yl]phenoxy}ethyl L-prolinate bis(trifluoroacetate) from Example 8 are dissolved in a mixture of dichloromethane and THF, and 24 mg (0.14 mmol) of p-toluenesulfonic acid are added. The precipitate formed is filtered off with suction, washed with diethyl ether and dried under high vacuum. This gives 42 mg (75% of theory) of the desired product as a colorless solid.

LC-MS (Method 2): Rₜ=2.09 min; MS (ESIpos): m/z=617 (M+H)⁺

¹H-NMR (400 MHz, DMSO-d₆): δ=1.85-2.04 (m, 3H), 2.22-2.32 (m, 1H), 2.28 (s, 3H), 3.16-3.30 (m, 2H), 4.32-4.63 (m, 5H), 4.65 (s, 2H), 7.08-7.14 (m, 4H), 7.44-7.52 (m, 4H), 7.57 (d, 2H), 7.93 (d, 2H), 7.96 (s, 1H), 8.14 (br. s, 2H), 8.88 (br. s, 1H), 9.40 (br. s, 1H).

### Example 11

### 2-{4-[2-Amino-6-({[2-(4-chlorophenyl)-1,3-thiazol-4-yl]methyl}thio)-3,5-dicyanopyridin-4-yl]-phenoxy}ethyl L-serinate hydrochloride

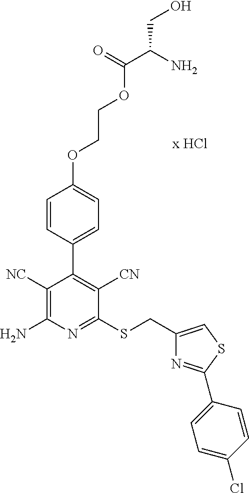

0.250 g (0.48 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.138 g (0.53 mmol) of Boc-L-serine, 0.111 g (0.58 mmol) of 1-(3-dimethylaminopropyl)-3-ethylcarbodiimide hydrochloride and 0.006 g (0.048 mmol) of 4-dimethylaminopyridine are combined in 10 ml of dichloromethane and 2.5 ml of DMF and stirred at room temperature for 24 hours. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated.

The residue is dissolved in 10 ml of dichloromethane, 2 ml of trifluoroacetic acid are added and the solution is stirred at room temperature overnight. The reaction mixture is then concentrated to dryness, and the residue is purified by preparative HPLC. 5 N of hydrochloric acid is added to the product fraction, and the mixture is concentrated. The residue is dissolved in THF, a 2 N solution of hydrogen chloride in dioxane is added and the mixture is concentrated again. This procedure is repeated once more. This gives 141 mg (44% of theory) of the desired product as a colorless powder.

LC-MS (Method 1): Rₜ=1.77 min; MS (ESIpos): m/z=607 (M+H)⁺

¹H-NMR (400 MHz, DMSO-d₆): δ=3.77-3.89 (m, 2H), 4.15-4.22 (m, 1H), 4.30-4.47 (m, 2H), 4.48-4.61 (m, 2H), 4.64 (s, 2H), 6.15 (br. s, 2H), 7.12 (d, 2H), 7.48 (d, 2H), 7.57 (d, 2H), 7.92 (d, 2H), 7.95 (s, 1H), 8.12 (br. s, 2H), 8.50 (s, 3H).

### Example 12

### 2-{4-[2-Amino-6-({[2-(4-chlorophenyl)-1,3-thiazol-4-yl]methyl}thio)-3,5-dicyanopyridin-4-yl]-phenoxy}ethyl L-glutaminate hydrochloride

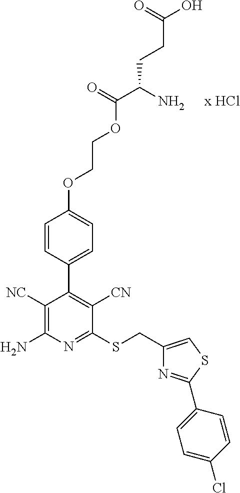

0.250 g (0.48 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.160 g (0.53 mmol) of Boc-L-glutamic acid, 0.111 g (0.58 mmol) of 1-(3-dimethylaminopropyl)-3-ethylcarbodiimide hydrochloride and 0.006 g (0.048 mmol) of 4-dimethylaminopyridine are combined in 10 ml of dichloromethane and 2.5 ml of DMF and stirred at room temperature for two hours. A clear solution is formed. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated.

The residue is dissolved in 10 ml of dichloromethane, 2 ml of trifluoroacetic acid are added and the solution is stirred at room temperature overnight. The reaction mixture is then concentrated to dryness, and the residue is purified by preparative HPLC. 5 N of hydrochloric acid is added to the product fraction, and the mixture is concentrated. The residue is dissolved in THF, a 2 N solution of hydrogen chloride in dioxane is added and the mixture is concentrated again. This procedure is repeated once more. This gives 233 mg (69% of theory) of the desired product as a colorless powder.

LC-MS (Method 1): Rₜ=1.90 min; MS (ESIpos): m/z=663 (M+H)⁺

¹H-NMR (400 MHz, DMSO-d₆): δ=2.04-2.10 (m, 2H), 4.08-4.14 (m, 1H), 4.30-4.36 (m, 2H), 4.47-4.60 (m, 2H), 4.64 (s, 2H), 5.78 (br. s, 2H), 7.12 (d, 2H), 7.48 (d, 2H), 7.57 (d, 2H), 7.94 (d, 2H), 7.96 (s, 1H), 8.13 (br. s, 2H), 8.60 (br. s, 2H).

### Example 13

### 2-{4-[2-Amino-6-({[2-(4-chlorophenyl)-1,3-thiazol-4-yl]methyl}thio)-3,5-dicyanopyridin-4-yl]-phenoxy}ethyl L-threoninate hydrochloride

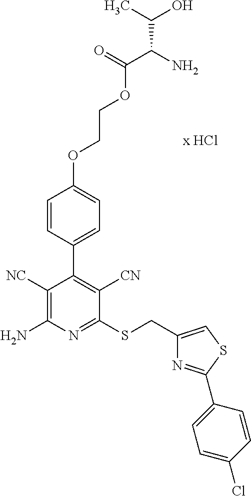

0.250 g (0.48 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.146 g (0.53 mmol) of Boc-L-threonine, 0.111 g (0.58 mmol) of 1-(3-dimethylaminopropyl)-3-ethylcarbodiimide hydrochloride and 0.006 g (0.048 mmol) of 4-dimethylaminopyridine are combined in 10 ml of dichloromethane and 2.5 ml of DMF and stirred at room temperature for two hours. A further 0.056 g (0.29 mmol) of 1-(3-dimethylaminopropyl)-3-ethylcarbodiimide hydrochloride and 0.003 g (0.024 mmol) of 4-dimethylaminopyridine are then added, and the mixture is once more stirred at room temperature overnight. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated.

The residue is dissolved in 10 ml of dichloromethane, 2 ml of trifluoroacetic acid are added and the solution is stirred at room temperature overnight. The reaction mixture is then concentrated to dryness, and the residue is dissolved in THF. The solution is poured into a mixture of semisaturated sodium bicarbonate solution and ethyl acetate. The organic phase is separated off, washed with saturated sodium chloride solution, dried over magnesium sulfate, filtered and concentrated. The residue is purified by preparative HPLC. 5 N of hydrochloric acid is added to the product fraction, and the mixture is concentrated. The residue is dissolved in THF, a 2 N solution of hydrogen chloride in dioxane is added and the mixture is concentrated again. This procedure is repeated once more. This gives 44 mg (14% of theory) of the desired product as a colorless powder.

LC-MS (Method 2): Rₜ=1.95 min; MS (ESIpos): m/z=621 (M+H)⁺

¹H-NMR (400 MHz, DMSO-d₆): δ=1.21 (d, 3H), 3.96-4.02 (m, 1H), 4.12-4.58 (m, 5H), 4.63 (s, 2H), 7.12 (d, 2H), 7.48 (d, 2H), 7.57 (d, 2H), 7.94 (d, 2H), 7.96 (s, 1H), 8.13 (br. s, 2H), 8.34 (br. s, 2H).

### Example 14

### 2-{4-[2-Amino-6-({[2-(4-chlorophenyl)-1,3-thiazol-4-yl]methyl}thio)-3,5-dicyanopyridin-4-yl]-phenoxy}ethyl L-lysinate dihydrochloride

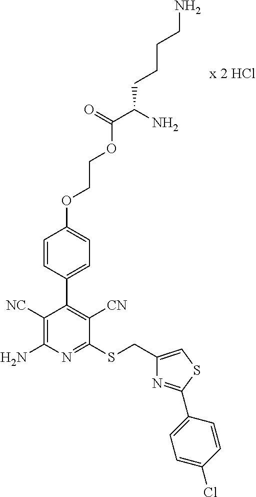

0.250 g (0.48 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.183 g (0.53 mmol) of N^{α},N^{ε}-di-Boc-L-lysine, 0.111 g (0.58 mmol) of 1-(3-dimethylaminopropyl)-3-ethylcarbodiimide hydrochloride and 0.006 g (0.048 mmol) of 4-dimethylaminopyridine are combined in 10 ml of dichloromethane and 2.5 ml of DMF and stirred at room temperature for two hours. A clear solution is formed. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated.

The residue is dissolved in 10 ml of dichloromethane, 2 ml of trifluoroacetic acid are added and the solution is stirred at room temperature overnight. The reaction mixture is then concentrated to dryness, and the residue is dissolved in THF. The reaction mixture is then poured into a mixture of semisaturated sodium bicarbonate solution and ethyl acetate. A precipitate is formed, which is filtered off with suction, washed with water and dried. The precipitate is then purified by column chromatography on silica gel (mobile phase: dichloromethane/methanol 10:1→dichloromethane/methanol/ammonia 100:20:2). The product fraction is dissolved in THF, and 1 ml of a 2 N solution of hydrogen chloride in dioxane are added. The precipitate formed is filtered off with suction, washed with THF and dried. This gives 235 mg (68% of theory) of the desired product as colorless crystals.

LC-MS (Method 1): Rₜ=1.53 min; MS (ESIpos): m/z=648 (M+H)⁺

¹H-NMR (400 MHz, DMSO-d₆): δ=1.36-1.60 (m, 4H), 1.74-1.86 (m, 2H), 2.68-2.78 (m, 2H), 4.02-4.62 (m, 5H), 4.64 (s, 2H), 7.12 (d, 2H), 7.48 (d, 2H), 7.57 (d, 2H), 7.92 (d, 2H), 7.95 (s, 1H), 8.13 (br. s, 2H), 8.55-8.65 (m, 2H).

### Example 15

### 2-{4-[2-Amino-6-({[2-(4-chlorophenyl)-1,3-thiazol-4-yl]methyl}thio)-3,5-dicyanopyridin-4-yl]-phenoxy}ethyl (N,N-dimethyl)glycinate

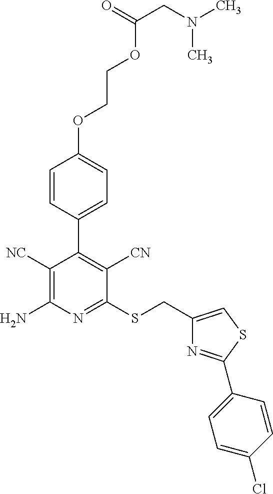

0.100 g (0.19 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.022 g (0.21 mmol) of N,N-dimethylglycine, 0.044 g (0.23 mmol) of 1-(3-dimethylaminopropyl)-3-ethylcarbodiimide hydrochloride and 0.002 g (0.019 mmol) of 4-dimethylaminopyridine are combined in 4 ml of dichloromethane and 1 ml of DMF and stirred at room temperature for two hours. A clear solution is formed. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated. The residue is purified by column chromatography on silica gel (mobile phase: dichloromethane/ethyl acetate 3:1→dichloromethane/ethyl acetate/methanol 30:10:1). This gives 82 mg (70% of theory) of the desired product as a colorless foam.

LC-MS (Method 1): Rₜ=1.87 min; MS (ESIpos): m/z=605 (M+H)⁺

¹H-NMR (400 MHz, DMSO-d₆): δ=2.22 (s, 6H), 3.21 (s, 2H), 4.27-4.30 (m, 2H), 4.40-4.44 (m, 2H), 4.62 (s, 2H), 7.11 (d, 2H), 7.47 (d, 2H), 7.57 (d, 2H), 7.93 (d, 2H), 7.96 (s, 1H), 8.13 (br. s, 2H).

### Example 16

### 2-{4-[2-Amino-6-({[2-(4-chlorophenyl)-1,3-thiazol-4-yl]methyl}thio)-3,5-dicyanopyridin-4-yl]-phenoxy}ethyl L-phenylalaninate hydrochloride

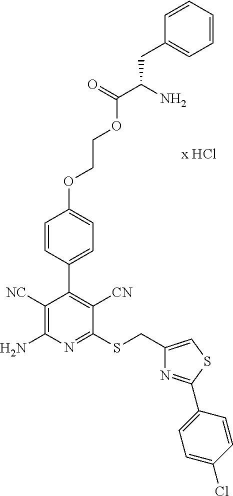

0.250 g (0.48 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.140 g (0.53 mmol) of Boc-L-phenylalanine, 0.111 g (0.58 mmol) of 1-(3-dimethylaminopropyl)-3-ethylcarbodiimide hydrochloride and 0.006 g (0.048 mmol) of 4-dimethylaminopyridine are combined in 10 ml of dichloromethane and 2.5 ml of DMF and stirred at room temperature for two hours. A clear solution is formed. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated.

The residue is dissolved in 10 ml of dichloromethane, 2 ml of trifluoroacetic acid are added and the solution is stirred at room temperature overnight. The reaction mixture is then concentrated to dryness, and the residue is crystallized from a mixture of dichloromethane and diethyl ether. The solid is filtered off with suction, washed with diethyl ether and dissolved in DMF. This solution is then poured into a mixture of semisaturated sodium bicarbonate solution and ethyl acetate. The organic phase is separated off, washed with saturated sodium chloride solution, dried over magnesium sulfate, filtered and concentrated. The residue crystallizes from a mixture of dichloromethane and diethyl ether. The crystals are filtered off with suction, washed with diethyl ether and dried. The residue is then purified by column chromatography on silica gel (mobile phase: dichloromethane/ethyl acetate 5:1→dichloromethane/ethyl acetate/methanol 100:20:2). The product fraction is dissolved in THF and 1 ml of a 2 N solution of hydrogen chloride in dioxane is added. The precipitate formed is filtered off with suction, washed with THF and dried. This gives 132 mg (38% of theory) of the desired product as colorless crystals.

LC-MS (Method 2): Rₜ=2.13 min; MS (ESIpos): m/z=667 (M+H)⁺

¹H-NMR (400 MHz, DMSO-d₆): δ=1.79 (br. s, 2H), 2.80-2.88 (m, 2H), 3.61 (t, 1H), 4.15-4.26 (m, 2H), 4.32-4.48 (m, 2H), 4.63 (s, 2H), 7.08-7.18 (m, 7H), 7.48 (d, 2H), 7.57 (d, 2H), 7.92 (d, 2H), 7.95 (s, 1H), 8.13 (br. s, 2H).

### Example 17

### 2-{4-[2-Amino-6-({[2-(4-chlorophenyl)-1,3-thiazol-4-yl]methyl}thio)-3,5-dicyanopyridin-4-yl]-phenoxy}ethyl L-phenylalaninate p-toluenesulfonic acid salt

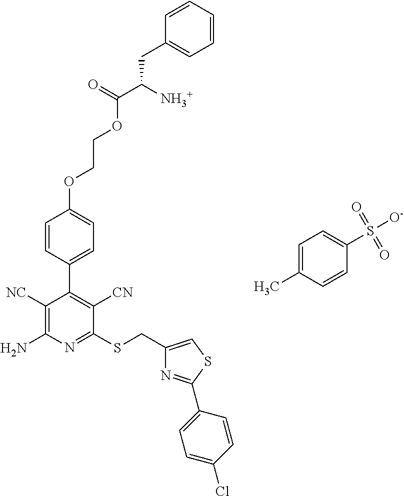

100 mg (0.15 mmol) of 2-{4-[2-amino-6-({[2-(4-chlorophenyl)-1,3-thiazol-4-yl]methyl}thio)-3,5-di-cyanopyridin-4-yl]phenoxy}ethyl L-phenylalaninate hydrochloride from Example 16 are dissolved in 2 ml of THF. 26 mg (0.15 mmol) of p-toluenesulfonic acid are added to the solution. The mixture is then concentrated to dryness. The residue is triturated with diethyl ether, and the solid is filtered off with suction. This gives 120 mg (95% of theory) of the desired product as a colorless powder.

LC-MS (Method 4): Rₜ=2.03 min; MS (ESIpos): m/z=667 (M+H)⁺

¹H-NMR (400 MHz, DMSO-d₆): δ=2.28 (s, 3H), 3.02-3.18 (m, 2H), 3.58 (br. s, 4H), 4.14-4.26 (m, 2H), 4.36-4.54 (m, 3H), 4.63 (s, 2H), 7.06-7.12 (m, 3H), 7.22 (s, 4H), 7.48 (d, 2H), 7.50 (d, 1H), 7.57 (d, 2H), 7.92 (d, 2H), 7.94 (s, 1H), 8.13 (br. s, 2H), 8.42 (br. s, 2H).

### Example 18

### 2-{4-[2-Amino-6-({[2-(4-chlorophenyl)-1,3-thiazol-4-yl]methyl}thio)-3,5-dicyanopyridin-4-yl]-phenoxy}ethyl L-phenylalaninate methanesulfonic acid salt

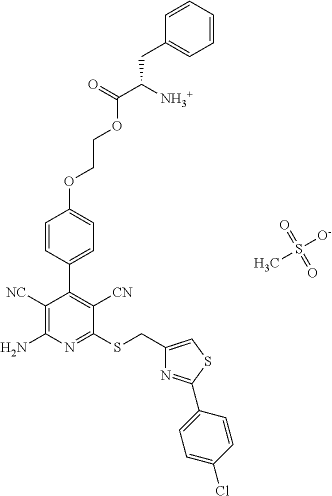

100 mg (0.15 mmol) of 2-{4-[2-amino-6-({[2-(4-chlorophenyl)-1,3-thiazol-4-yl]methyl}thio)-3,5-di-cyanopyridin-4-yl]phenoxy}ethyl L-phenylalaninate hydrochloride from Example 16 are dissolved in 2 ml of THF. 14 mg (0.15 mmol) of methanesulfonic acid are added to the solution. The precipitate formed is filtered off with suction, washed with diethyl ether and dried. This gives 104 mg (91% of theory) of the desired product as a colorless powder.

LC-MS (Method 4): Rₜ=2.03 min; MS (ESIpos): m/z=667 (M+H)⁺

¹H-NMR (400 MHz, DMSO-d₆): δ=2.30 (s, 3H), 3.02-3.18 (m, 2H), 3.72 (br. s, 4H), 4.15-4.27 (m, 2H), 4.35-4.55 (m, 3H), 4.63 (s, 2H), 7.08 (d, 2H), 7.21 (s, 5H), 7.48 (d, 2H), 7.57 (d, 2H), 7.92 (d, 2H), 7.95 (s, 1H), 8.13 (br. s, 2H), 8.42 (br. s, 2H).

### Example 19

### 4-(2-{4-[2-Amino-6-({[2-(4-chlorophenyl)-1,3-thiazol-4-yl]methyl}thio)-3,5-dicyanopyridin-4-yl]-phenoxy}ethoxy)-4-oxobutanoic acid hydrochloride

### Step a)

### 2-{4-[2-Amino-6-({[2-(4-chlorophenyl)-1,3-thiazol-4-yl]methyl}thio)-3,5-dicyanopyridin-4-yl]-phenoxy}ethyl tert-butyl succinate

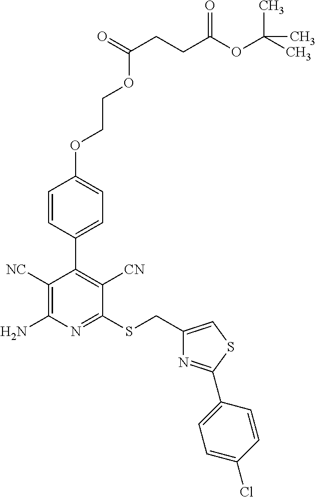

2.50 g (4.81 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.92 g (5.29 mmol) of mono-tert-butyl succinate, 1.11 g (5.77 mmol) of 1-(3-dimethylaminopropyl)-3-ethylcarbodiimide hydrochloride and 0.059 g (0.048 mmol) of 4-dimethylaminopyridine are combined in 100 ml of dichloromethane and 25 ml of DMF and stirred at room temperature overnight. The reaction mixture is then poured into a mixture of semisaturated ammonium chloride solution and ethyl acetate. The organic phase is separated off, washed successively with water, saturated sodium bicarbonate solution and saturated sodium chloride solution, dried over magnesium sulfate, filtered and concentrated. The residue is triturated with diethyl ether, and the solid is filtered off with suction, washed with diethyl ether and dried. This gives 2.76 g (85% of theory) of the desired product as colorless crystals.

LC-MS (Method 1): Rₜ=3.06 min; MS (ESIpos): m/z=676 (M+H)⁺

¹H-NMR (400 MHz, DMSO-d₆): δ=1.38 (s, 9H), 2.44-2.55 (m, 4H), 4.25-4.28 (m, 2H), 4.35-4.40 (m, 2H), 4.63 (s, 2H), 7.12 (d, 2H), 7.47 (d, 2H), 7.58 (d, 2H), 7.94 (d, 2H), 7.96 (s, 1H), 8.13 (br. s, 2H).

### Step b)

### 4-(2-{4-[2-Amino-6-({[2-(4-chlorophenyl)-1,3-thiazol-4-yl]methyl}thio)-3,5-dicyanopyridin-4-yl]-phenoxy}ethoxy)-4-oxobutanoic acid hydrochloride

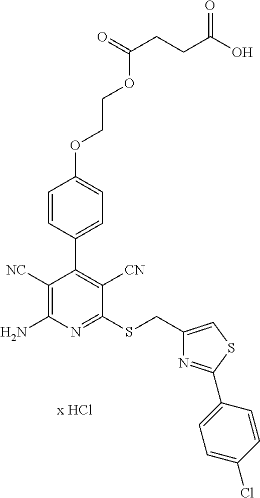

845 mg (1.25 mmol) of 2-{4-[2-amino-6-({[2-(4-chlorophenyl)-1,3-thiazol-4-yl]methyl}thio)-3,5-di-cyanopyridin-4-yl]phenoxy}ethyl tert-butyl succinate and 5 ml of trifluoroacetic acid are combined in 50 ml of dichloromethane and stirred at room temperature for two hours. The reaction mixture is then concentrated, and twice toluene is added and the mixture is concentrated again. The residue crystallizes from dichloromethane. The crystals are filtered off with suction, washed with diethyl ether and then dissolved in a mixture of dichloromethane and THF. 5 ml of a 2 N solution of hydrogen chloride in diethyl ether are added. The precipitate formed is filtered off with suction, washed with diethyl ether and dried. This gives 725 mg (88% of theory) of the desired product as a colorless powder.

LC-MS (Method 2): Rₜ=2.75 min; MS (ESIpos): m/z=620 (M+H)⁺

¹H-NMR (400 MHz, DMSO-d₆): δ=2.45-2.58 (m, 4H), 4.25-4.29 (m, 2H), 4.43-4.48 (m, 2H), 4.65 (s, 2H), 7.12 (d, 2H), 7.47 (d, 2H), 7.56 (d, 2H), 7.94 (d, 2H), 7.95 (s, 1H), 8.13 (br. s, 2H).

### Example 20

### 4-(2-{4-[2-Amino-6-({[2-(4-chlorophenyl)-1,3-thiazol-4-yl]methyl}thio)-3,5-dicyanopyridin-4-yl]-phenoxy}ethoxy)-4-oxobutanoic acid potassium salt

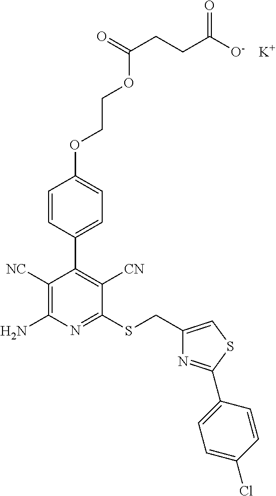

500 mg (0.81 mmol) of 4-(2-{4-[2-amino-6-({[2-(4-chlorophenyl)-1,3-thiazol-4-yl]methyl}thio)-3,5-dicyanopyridin-4-yl]phenoxy}ethoxy)-4-oxobutanoic acid from Example 19 are dissolved in 5 ml of THF. 5 ml of water and 45 mg (0.81 mmol) of potassium hydroxide are added. The THF is then removed on a rotary evaporator, and the solution that remains is freeze-dried. This gives 532 mg (100% of theory) of the desired product as a colorless powder.

LC-MS (Method 1): Rₜ=2.59 min; MS (ESIpos): m/z=620 (M+H)⁺

¹H-NMR (400 MHz, DMSO-d₆): δ=2.12 (t, 2H), 2.38 (t, 2H), 4.25-4.28 (m, 2H), 4.28-4.34 (m, 2H), 4.64 (s, 2H), 7.12 (d, 2H), 7.48 (d, 2H), 7.58 (d, 2H), 7.94 (d, 2H), 7.95 (s, 1H), 8.13 (br. s, 2H).

### Example 21

### 4-(2-{4-[2-Amino-6-({[2-(4-chlorophenyl)-1,3-thiazol-4-yl]methyl}thio)-3,5-dicyanopyridin-4-yl]-phenoxy}ethoxy)-4-oxobutanoic acid sodium salt

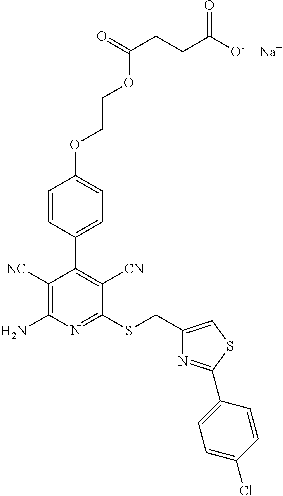

500 mg (0.81 mmol) of 4-(2-{4-[2-amino-6-({[2-(4-chlorophenyl)-1,3-thiazol-4-yl]methyl}thio)-3,5-dicyanopyridin-4-yl]phenoxy}ethoxy)-4-oxobutanoic acid from Example 19 are dissolved in 5 ml of THF. 5 ml of water and 32 mg (0.81 mmol) of sodium hydroxide are added. The THF is then removed on a rotary evaporator, and the solution that remains is freeze-dried. This gives 520 mg (100% of theory) of the desired product as a colorless powder.

LC-MS (Method 1): Rₜ=2.60 min; MS (ESIpos): m/z=620 (M+H)⁺

¹H-NMR (400 MHz, DMSO-d₆): δ=2.09 (t, 2H), 2.38 (t, 2H), 4.24-4.34 (m, 4H), 4.64 (s, 2H), 7.12 (d, 2H), 7.47 (d, 2H), 7.57 (d, 2H), 7.92 (d, 2H), 7.95 (s, 1H), 8.13 (br. s, 2H).

### Example 22

### 2-{4-[2-Amino-6-({[2-(4-chlorophenyl)-1,3-thiazol-4-yl]methyl}thio)-3,5-dicyanopyridin-4-yl]-phenoxy}ethyl L-ornithinate dihydrochloride

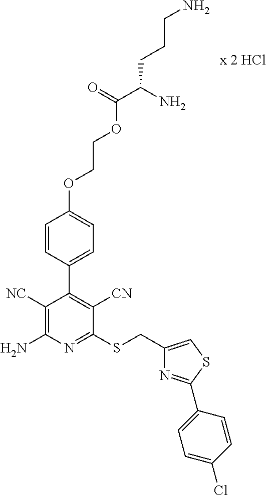

1 g (1.92 mmol) of 2-amino-6-({[2-(4-chlorophenyl)-1,3-thiazol-4-yl]methyl}thio)-4-[4-(2-hydroxy-ethoxy)phenyl]pyridine-3,5-dicarbonitrile, 1.92 g (5.77 mmol) of N^{α},N^{δ}-di-Boc-L-ornithine, 0.442 g (2.31 mmol) of 1-(3-dimethylaminopropyl)-3-ethylcarbodiimide hydrochloride and 0.117 g (0.96 mmol) of 4-dimethylaminopyridine are combined in 25 ml of dichloromethane and 25 ml of DMF and stirred at room temperature for one hour. A clear solution is formed. The reaction mixture is then concentrated, and the residue is taken up in dichloromethane and extracted successively with 5% strength citric acid, sodium bicarbonate solution and water. The organic phase is concentrated, and the residue is purified by flash chromatography on silica gel using the mobile phase dichloromethane/ethyl acetate (5:1). The appropriate fractions are combined, and the solvent is removed under reduced pressure. After the residue has been dried under high vacuum, 1.6 g (99% of theory) of the protected intermediate remain.

The residue is taken up in 30 ml of dichloromethane and 20 ml of anhydrous trifluoroacetic acid, and the solution is stirred at room temperature for 30 min. The reaction mixture is then concentrated to dryness, and the residue is concentrated two more times from acetonitrile. A 2 M solution of hydrogen chloride in diethyl ether is then added to the residue that remains. The precipitate formed is filtered off with suction and washed with diethyl ether. The precipitate is then recrystallized from 25 ml of methanol. This gives 744 mg (55% of theory) of the desired product as colorless crystals.

HPLC (Method 7): Rₜ=4.9 min;

LC-MS (Method 12): Rₜ=1.32 min; MS (ESIpos): m/z=634 (M+H)⁺

¹H-NMR (400 MHz, DMSO-d₆): δ=1.6-2.0 (m, 4H), 2.8 (t, 2H), 4.1 (t, 1H), 4.35 (m, 2H), 4.55 (m, 2H), 4.65 (s, 2H), 7.12 (d, 2H), 7.48 (d, 2H), 7.57 (d, 2H), 7.92 (d, 2H), 7.95 (s, 1H), 8.0 (br. s, 2H), 8.55-8.65 (m, 2H).

### Example 23

### 2-{4-[2-Amino-6-({[2-(4-chlorophenyl)-1,3-thiazol-4-yl]methyl}thio)-3,5-dicyanopyridin-4-yl]-phenoxy}ethyl L-valinate dihydrochloride

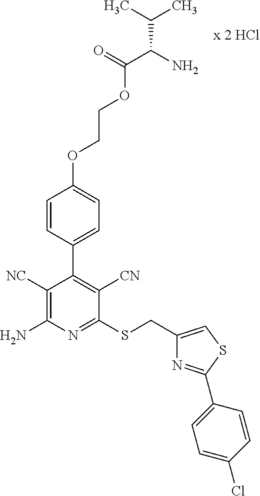

1 g (1.92 mmol) of 2-amino-6-({[2-(4-chlorophenyl)-1,3-thiazol-4-yl]methyl}thio)-4-[4-(2-hydroxy-ethoxy)phenyl]pyridine-3,5-dicarbonitrile, 0.460 g (2.11 mmol) of Boc-L-valine, 0.442 g (2.31 mmol) of 1-(3-dimethylaminopropyl)-3-ethylcarbodiimide hydrochloride and 0.023 g (0.19 mmol) of 4-dimethylaminopyridine are combined in 40 ml of dichloromethane and 10 ml of DMF and stirred at room temperature overnight. A clear solution is formed. The reaction mixture is then poured into a mixture of semisaturated ammonium chloride solution and dichloromethane. The organic phase is separated off, washed successively with water, saturated sodium bicarbonate solution and saturated sodium chloride solution, dried over magnesium sulfate, filtered and concentrated. The residue is purified by flash chromatography on silica gel using the mobile phase dichloromethane/ethyl acetate (gradient 10:1→7:1→5:1). The appropriate fractions are combined, and the solvent is removed under reduced pressure. After the residue has been dried under high vacuum, 0.85 g (62% of theory) of the protected intermediate remain.

The residue is taken up in 5 ml of dichloromethane and 5 ml of anhydrous trifluoroacetic acid, and the solution is stirred at room temperature for 2 h. The reaction mixture is then concentrated to dryness, and the residue is two more times concentrated with toluene. The residue that remains is taken up in acetonitrile, and 5 ml of 1 N hydrochloric acid are added. The precipitate formed is filtered off with suction and washed with isopropanol and diethyl ether. This gives 673 mg (82% of theory) of the title compound as colorless crystals.

HPLC (Method 7): Rₜ=5.3 min;

LC-MS (Method 10): Rₜ=2.01 min; MS (ESIpos): m/z=619 (M+H)⁺.

### Example 24

### 2-{4-[2-Amino-6-({[2-(4-chlorophenyl)-1,3-thiazol-4-yl]methyl}thio)-3,5-dicyanopyridin-4-yl]-phenoxy}ethyl (2S)-2,4-diaminobutanoate dihydrochloride

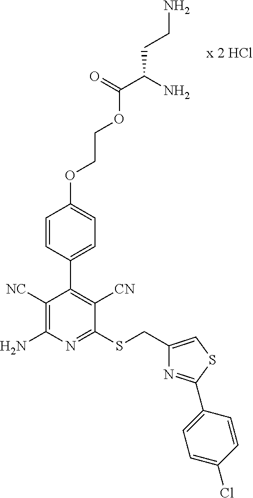

1 g (1.92 mmol) of 2-amino-6-({[2-(4-chlorophenyl)-1,3-thiazol-4-yl]methyl}thio)-4-[4-(2-hydroxy-ethoxy)phenyl]pyridine-3,5-dicarbonitrile, 0.67 g (2.1 mmol) of N^{α},N^{γ}-di-Boc-L-diaminobutyric acid, 0.442 g (2.31 mmol) of 1-(3-dimethylaminopropyl)-3-ethylcarbodiimide hydrochloride and 0.023 g (0.19 mmol) of 4-dimethylaminopyridine are combined in 25 ml of dichloromethane and 6 ml of DMF and stirred at room temperature overnight. A clear solution is formed. The reaction mixture is then poured into a mixture of semisaturated ammonium chloride solution and dichloromethane. The organic phase is separated off, washed successively with water, saturated sodium bicarbonate solution and saturated sodium chloride solution, dried over magnesium sulfate, filtered and concentrated. The residue is purified by flash chromatography on silica gel using the mobile phase dichloromethane/ethyl acetate (gradient 10:1→6:1→3:1). The column is then eluted with dichloromethane/ethyl acetate/methanol (150:50:5). The appropriate fractions are concentrated and the residue that remains is purified further by preparative HPLC (Method 5). The product fractions are combined, and the solvent is removed under reduced pressure. After the residue has been dried under high vacuum, 0.542 g (33% of theory) of the protected intermediate remains.

The residue is taken up in 3 ml of dichloromethane and 3 ml of anhydrous trifluoroacetic acid, and the solution is stirred at room temperature for 1 h. The reaction mixture is then concentrated to dryness, and the residue once more concentrated with dichloromethane. The residue that remains is then taken up in 150 ml of ethyl acetate, and a saturated solution of hydrogen chloride in diethyl ether is added. The precipitate formed is filtered off with suction, twice washed with diethyl ether and then dried. The mixture is then lyophilized from water. This gives 415 mg (92% of theory) of the title compound.

HPLC (Method 7): Rₜ=4.9 min;

LC-MS (Method 12): Rₜ=1.4 min; MS (ESIpos): m/z=620 (M+H)⁺.

### Example 25

### 2-(4-{2-[(4-Aminobutanoyl)amino]-6-({[2-(4-chlorophenyl)-1,3-thiazol-4-yl]methyl}thio)-3,5-di-cyanopyridin-4-yl}phenoxy)ethyl 4-aminobutanoate dihydrochloride

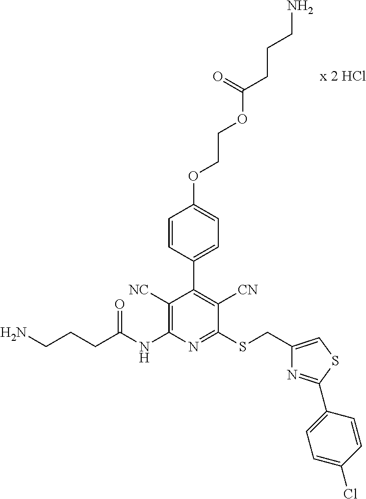

1.5 g (2.88 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 1.41 g (6.92 mmol) of 4-[(tert-butoxycarbonyl)-amino]butyric acid, 1.3 g (6.92 mmol) of 1-(3-dimethylaminopropyl)-3-ethylcarbodiimide hydrochloride and 0.846 g (6.92 mmol) of 4-dimethylaminopyridine are combined in 90 ml of dichloromethane and stirred under reflux overnight. The reaction mixture is then poured into a mixture of semisaturated ammonium chloride solution and dichloromethane. The organic phase is separated off, washed successively with saturated sodium bicarbonate solution and saturated sodium chloride solution, dried over magnesium sulfate, filtered and concentrated. The residue is purified by flash chromatography on silica gel using the mobile phase dichloromethane/ethyl acetate (gradient 10:1→7:1→5:1→3:1→2:1). The appropriate fractions are combined and the solvent is removed. The residue that remains is dissolved in dichloromethane and reprecipitated using a mixture of diethyl ether and petroleum ether. After drying under high vacuum, 1360 mg (53% of theory) of the protected intermediate remain.

The residue is taken up in 10 ml of dichloromethane and 5 ml of anhydrous trifluoroacetic acid, and die solution is stirred at room temperature for 1 h. The reaction mixture is then concentrated to dryness, and the residue is two more times concentrated with toluene. The residue that remains is then taken up in 15 ml of dichloromethane, 5 ml of THF and 5 ml of methanol, and a saturated solution of hydrochloride in diethyl ether is added. The precipitate formed is filtered off with suction, twice washed with diethyl ether and dried. The mixture is then lyophilized from water. This gives 1170 mg (95% of theory) of the title compound.

HPLC (Method 7): Rₜ=4.7 min;

LC-MS (Method 11): Rₜ=1.1 min; MS (ESIpos): m/z=690 (M+H)⁺.

### Example 26

### 2-{4-[2-(beta-Alanylamino)-6-({[2-(4-chlorophenyl)-1,3-thiazol-4-yl]methyl}thio)-3,5-dicyano-pyridin-4-yl]phenoxy}ethyl beta-alaninate dihydrochloride

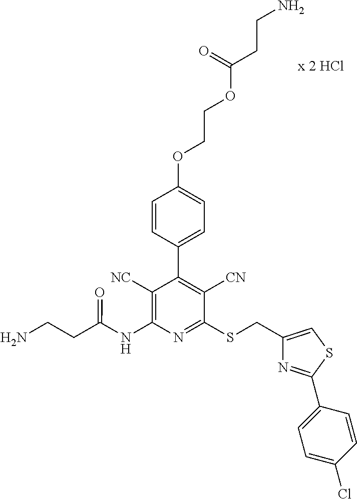

The title compound is prepared analogously to Example 25 from 1 g (1.92 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile and 0.8 g (4.23 mmol) of N-(tert-butoxycarbonyl)-β-alanine.

Yield: 61% of theory over the two steps.

HPLC (Method 7): Rₜ=4.7 min;

LC-MS (Method 4): Rₜ=1.51 min; MS (ESIpos): m/z=662 (M+H)⁺.

### Example 27

### 2-Hydroxy-N-(2-hydroxyethyl)ethanaminium 4-(2-{4-[2-amino-6-({[2-(4-chlorophenyl)-1,3-thiazol-4-yl]methyl}thio)-3,5-dicyanopyridin-4-yl]phenoxy}ethoxy) 4-oxobutanoate

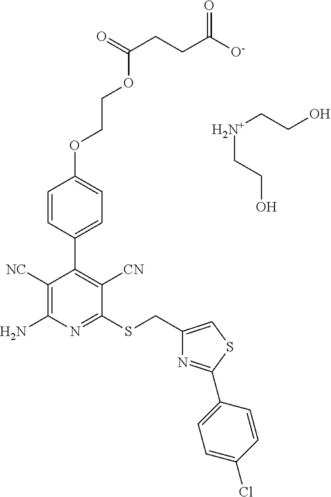

8450 mg (12.5 mmol) of 2-{4-[2-amino-6-({[2-(4-chlorophenyl)-1,3-thiazol-4-yl]methyl}thio)-3,5-dicyanopyridin-4-yl]phenoxy}ethyl tert-butyl succinate (Example 19, Step a) and 50 ml of trifluoroacetic acid are combined in 500 ml of dichloromethane and stirred at room temperature for two hours. The reaction mixture is then concentrated, and twice toluene is added and the mixture is concentrated again. The residue crystallizes from dichloromethane. The crystals are filtered off with suction and washed with diethyl ether.

An aliquot of 3380 mg (5.45 mmol) of the carboxylic acid obtained is taken up in 360 ml of isopropanol, and 573 mg (5.45 mmol) of diethanolamine and 60 ml of water are added. The iso-propanol is evaporated under reduced pressure and the aqueous solution is then lyophilized. In this manner, 3850 mg of the title compound are obtained.

LC-MS (Method 8): Rₜ=3.99 min; MS (ESIpos): m/z=620 (M+H)⁺.

### Example 28

### 2-Hydroxy-N,N,N-trimethylethanaminium 4-(2-{4-[2-amino-6-({[2-(4-chlorophenyl)-1,3-thiazol-4-yl]methyl}thio)-3,5-dicyanopyridin-4-yl]phenoxy}ethoxy) 4-oxobutanoate

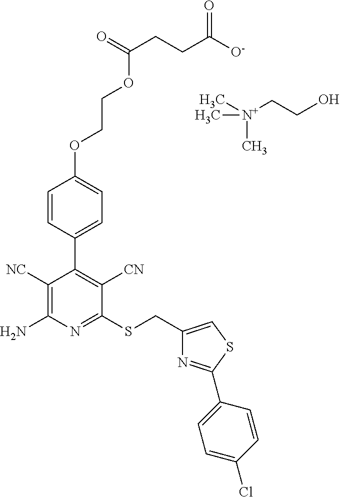

8450 mg (12.5 mmol) of 2-{4-[2-amino-6-({[2-(4-chlorophenyl)-1,3-thiazol-4-yl]methyl}thio)-3,5-dicyanopyridin-4-yl]phenoxy}ethyl tert-butyl succinate (Example 19, Step a) and 50 ml of trifluoroacetic acid are combined in 500 ml of dichloromethane and stirred at room temperature for two hours. The reaction mixture is then concentrated, and twice toluene is added and the mixture is concentrated again. The residue crystallizes from dichloromethane. The crystals are filtered off with suction and washed with diethyl ether.

An aliquot of 375 mg (0.605 mmol) of the carboxylic acid obtained is taken up in 13 ml of dioxane, and 13.2 ml of an aqueous choline solution (0.605 mmol) are added. After brief stirring at RT, the solution is lyophilized. This gives 437 mg (quant.) of the title compound.

LC-MS (Method 8): Rₜ=3.82 min; MS (ESIpos): m/z=620 (M+H)⁺.

### Example 29

### 2-Hydroxy-N,N,N-trimethylethanaminium 3-(2-{4-[2-amino-6-({[2-(4-chlorophenyl)-1,3-thiazol-4-yl]methyl}thio)-3,5-dicyanopyridin-4-yl]phenoxy}ethoxy) 3-oxopropanoate

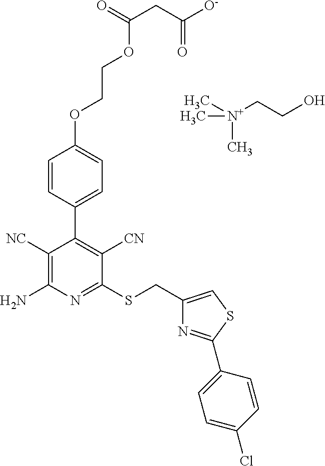

### Step a):

1 g (1.92 mmol) of 2-amino-6-({[2-(4-chlorophenyl)-1,3-thiazol-4-yl]methyl}thio)-4-[4-(2-hydroxy-ethoxy)phenyl]pyridine-3,5-dicarbonitrile, 0.389 g (2.12 mmol) of mono-tert-butyl malonate, 0.442 g (2.31 mmol) of 1-(3-dimethylaminopropyl)-3-ethylcarbodiimide hydrochloride and 0.023 g (0.19 mmol) of 4-dimethylaminopyridine are combined in 40 ml of dichloromethane and 10 ml of DMF and stirred at room temperature overnight. The reaction mixture is then poured into a mixture of semisaturated ammonium chloride solution and dichloromethane. The organic phase is separated off, washed successively with water, saturated sodium bicarbonate solution and saturated sodium chloride solution, dried over magnesium sulfate, filtered and concentrated. The residue is dissolved in dichloromethane, reprecipitated with petroleum ether, filtered off with suction and then purified by flash chromatography on silica gel using the mobile phase dichloromethane/ethyl acetate (gradient 10:1→7:1→5:1). The appropriate fractions are combined, and the solvent is removed under reduced pressure. After the residue has been dried under high vacuum, 0.916 g (72% of theory) of the protected intermediate remain.

LC-MS (Method 10): Rₜ=3.24 min; MS (ESIpos): m/z=662 (M+H)⁺.

### Step b):

916 mg (1.38 mmol) of the intermediate from Step a) are taken up in 5 ml of dichloromethane, 5 ml of trifluoroacetic acid are added dropwise and the mixture is stirred at room temperature for two hours. The reaction mixture is then concentrated, and twice toluene is added and the mixture is concentrated again. The residue is taken up in dichloromethane, and isopropanol is added. The precipitated crystals are filtered off with suction, washed with diethyl ether and then dried under high vacuum. This gives 543 mg (65% of theory) of the free acid as a colorless powder.

HPLC (Method 7): Rₜ=5.7 min;

LC-MS (Method 10): Rₜ=2.89 min; MS (ESIpos): m/z=606 (M+H)⁺.

### Step c):

An aliquot of 100 mg (0.165 mmol) of the carboxylic acid from Step b) is taken up in 3.5 ml of dioxane, and 3.6 ml of an aqueous choline solution (0.165 mmol) are added. After brief stirring at RT, the solution is lyophilized. This gives 116 mg (quant.) of the title compound.

LC-MS (Method 8): Rₜ=3.79 min; MS (ESIpos): m/z=606 (M+H)⁺.

### Example 30

### 2-Hydroxy-N-(2-hydroxyethyl)ethanaminium 3-(2-{4-[2-amino-6-({[2-(4-chlorophenyl)-1,3-thiazol-4-yl]methyl}thio)-3,5-dicyanopyridin-4-yl]phenoxy}ethoxy)-3-oxopropanoate

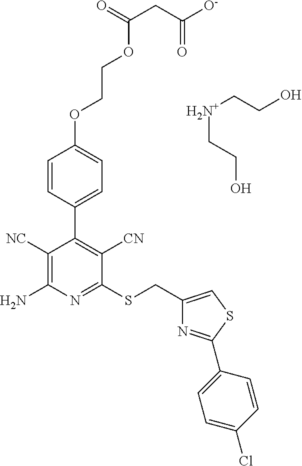

An aliquot of 100 mg (0.165 mmol) of the carboxylic acid from Example 29, Step b) is taken up in 3.5 ml of dioxane, and 2 ml of an aqueous diethanolamine solution (0.165 mmol) are added. After brief stirring at RT, the solution is lyophilized. This gives 118 mg (quant.) of the title compound.

LC-MS (Method 8): Rₜ=3.79 min; MS (ESIpos): m/z=606 (M+H)⁺.

### Example 31

### 2-Hydroxy-N,N,N-trimethylethanaminium (2Z)-4-(2-{4-[2-amino-6-({[2-(4-chlorophenyl)-1,3-thiazol-4-yl]methyl}thio)-3,5-dicyanopyridin-4-yl]phenoxy}ethoxy)-4-oxobut-2-enoate

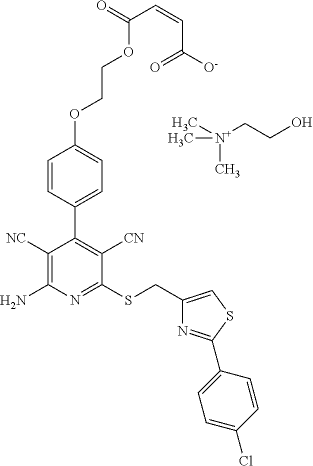

### Step a):

3 g (5.77 mmol) of 2-amino-6-({[2-(4-chlorophenyl)-1,3-thiazol-4-yl]methyl}thio)-4-[4-(2-hydroxy-ethoxy)phenyl]pyridine-3,5-dicarbonitrile and 3.39 g (34.6 mmol) of maleic anhydride are combined in 200 ml of pyridine, and the mixture is stirred at 110° C. for 6 h. Another 1.7 g of maleic anhydride are then added, and the reaction mixture is stirred at 110° C. for 3 h. The reaction mixture is then cooled and concentrated under high vacuum. The residue is taken up in dichloromethane/methanol and passed through a frit filled with silica gel. The filter cake is washed with 1.5 litres of dichloromethane/methanol (1:1), and the filtrate is then concentrated. The residue is purified by flash chromatography on silica gel using the mobile phase toluene/ethanol (2:1). The appropriate fractions are combined, and the solvent is removed under reduced pressure. The residue is then purified once more by chromatography on silica gel using the mobile phase toluene/ethanol (7:1). Once more, the product fractions are combined and concentrated. After drying under high vacuum, 1018 mg (29% of theory) of the desired maleic semiester remain.

HPLC (Method 7): Rₜ=5.83 min;

LC-MS (Method 8): Rₜ=3.88 min; MS (ESIpos): m/z=618 (M+H)⁺.

### Step b):

An aliquot of 29 mg (0.047 mmol) of the compound from Step a) is taken up in 15 ml of dioxane, and 469 μl of a 0.1 M aqueous choline solution (0.047 mmol) are added. After brief stirring at RT, the solution is lyophilized. This gives 27 mg (81% of theory) of the title compound.

LC-MS (Method 10): Rₜ=3.02 min; MS (ESIpos): m/z=618 (M+H)⁺.

### Example 32

### 2-Hydroxy-N-(2-hydroxyethyl)ethanaminium (2Z)-4-(2-{4-[2-amino-6-({[2-(4-chlorophenyl)-1,3-thiazol-4-yl]methyl}thio)-3,5-dicyanopyridin-4-yl]phenoxy}ethoxy)-4-oxobut-2-enoate

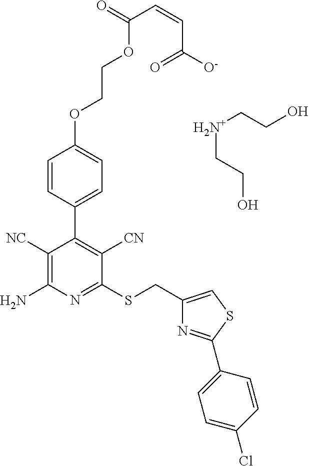

An aliquot of 29 mg (0.047 mmol) of the compound from Example 31, Step a) is taken up in 15 ml of dioxane, and 469 μl of an aqueous diethanolamine solution (0.047 mmol) are added. After brief stirring at RT, the solution is lyophilized. This gives 31 mg (91% of theory) of the title compound.

LC-MS (Method 10): Rₜ=3.09 min; MS (ESIpos): m/z=618 (M+H)⁺.

### Example 33

### 2-{4-[2-Amino-6-({[2-(4-chlorophenyl)-1,3-thiazol-4-yl]methyl}thio)-3,5-dicyanopyridin-4-yl]-phenoxy}ethyl L-histidinate dihydrochloride

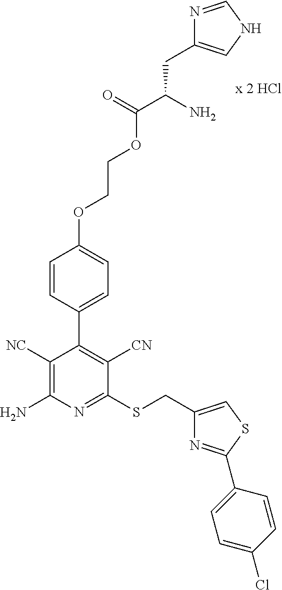

0.5 g (0.961 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 1.025 g (2.884 mmol) of N,1-bis(tert-butoxy-carbonyl)-L-histidine, 0.24 g (1.25 mmol) of 1-(3-dimethylaminopropyl)-3-ethylcarbodiimide hydrochloride and 0.059 g (0.481 mmol) of 4-dimethylaminopyridine are combined in 25 ml of dichloromethane and 25 ml of DMF and stirred at room temperature overnight. Another 0.12 g of 1-(3-dimethylaminopropyl)-3-ethylcarbodiimide hydrochloride and 0.01 g of 4-dimethylaminopyridine are then added, and stirring at RT is continued for 16 h. The reaction mixture is then concentrated. The residue is taken up in dichloromethane and extracted successively with 5% strength citric acid, sodium bicarbonate solution and water. The organic phase is concentrated, and the residue is purified by flash chromatography on silica gel using the mobile phase toluene/ethanol (6:1). The appropriate fractions are combined, and the solvent is removed under reduced pressure. After the residue has been dried under high vacuum, 653 mg (79% of theory) of the protected intermediate remain.

This intermediate is taken up in 25 ml of dichloromethane and 20 ml of anhydrous trifluoroacetic acid, and the solution is stirred at room temperature for 30 min. The reaction mixture is then concentrated to dryness, and two more times the residue is evaporated with acetonitrile. A 2 M solution of hydrogen chloride in diethyl ether is then added to the residue that remains. The precipitate formed is filtered off with suction, washed with diethyl ether and dried under high vacuum. This gives 556 mg (quant.) of the title compound as colorless crystals.

HPLC (Method 7): Rₜ=4.8 min;

LC-MS (Method 10): Rₜ=1.63 min; MS (ESIpos): m/z=654 (M+H)⁺.

### Example 34

### 2-(4-{2-({[2-(4-Chlorophenyl)-1,3-thiazol-4-yl]methyl}thio)-3,5-dicyano-6-[(N,N-dimethylglycyl)-amino]pyridin-4-yl}phenoxy)ethyl N,N-dimethylglycinate

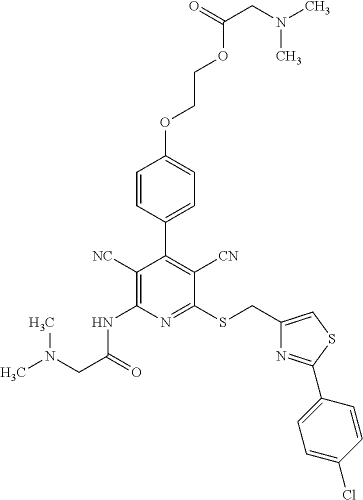

1 g (1.92 mmol) of 2-amino-6-({[2-(4-chlorophenyl)-1,3-thiazol-4-yl]methyl}thio)-4-[4-(2-hydroxy-ethoxy)phenyl]pyridine-3,5-dicarbonitrile, 436 mg (4.23 mmol) of N,N-dimethylglycine hydrochloride, 885 mg (4.6 mmol) of 1-(3-dimethylaminopropyl)-3-ethylcarbodiimide hydrochloride and 564 mg (4.62 mmol) of 4-dimethylaminopyridine are combined in 100 ml of dichloromethane and heated under reflux overnight. A further 218 mg (2.12 mmol) of N,N-dimethylglycine, 442 mg (2.3 mmol) of 1-(3-dimethylaminopropyl)-3-ethylcarbodiimide hydrochloride and 282 mg (2.32 mmol) of 4-dimethylaminopyridine are then added, and the reaction mixture is once more heated under reflux overnight. The reaction mixture is then poured into a mixture of semisaturated sodium bicarbonate solution and dichloromethane. The organic phase is separated off, washed with saturated sodium chloride solution, dried over magnesium sulfate, filtered and concentrated. The residue is purified by flash chromatography on silica gel using, as mobile phase, initially dichloromethane/ethyl acetate (3:1) and then dichloromethane/ethyl acetate/methanol (150:50:10). The appropriate fractions are combined, and the solvent is removed under reduced pressure. The residue is then purified further by flash chromatography on silica gel using the mobile phase toluene/ethyl acetate (2:1). Once more, the product fractions are combined and concentrated. After drying of the residue under high vacuum, 1.04 g (78% of theory) of the title compound are obtained as a colorless foam.

LC-MS (Method 12): Rₜ=1.3 min; MS (ESIpos): m/z=690 (M+H)⁺.

### Example 35

### 2-(4-{2-({[2-(4-Chlorophenyl)-1,3-thiazol-4-yl]methyl}thio)-3,5-dicyano-6-[(N,N-dimethylglycyl)-amino]pyridin-4-yl}phenoxy)ethyl N,N-dimethylglycinate dihydrochloride

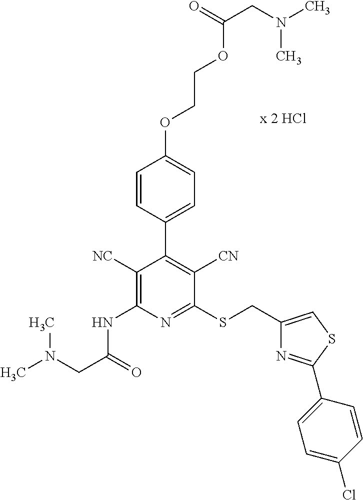

250 mg (0.362 mmol) of the compound from Example 34 are suspended in 10 ml of 1 N hydrochloric acid, and 2 ml of acetonitrile are added. After brief stirring, a clear solution is formed which is then lyophilized. This gives 275 mg (99% of theory) of the title compound as a colorless foam.

LC-MS (Method 12): Rₜ=1.15 min; MS (ESIpos): m/z=690 (M+H)⁺.

### Example 36

### (2S)-2-Amino-5-(2-{4-[2-amino-6-({[2-(4-chlorophenyl)-1,3-thiazol-4-yl]methyl}thio)-3,5-dicyano-pyridin-4-yl]phenoxy}ethoxy)-5-oxopentanoic acid dihydrochloride

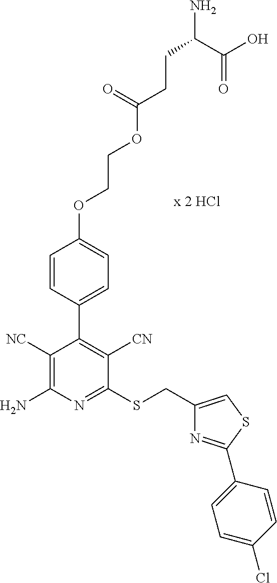

3.117 g (6 mmol) of 2-amino-6-({[2-(4-chlorophenyl)-1,3-thiazol-4-yl]methyl}thio)-4-[4-(2-hydroxy-ethoxy)phenyl]pyridine-3,5-dicarbonitrile, 2.0 g (6.59 mmol) of (4S)-5-tert-butoxy-4-[(tert-butoxy-carbonyl)amino]-5-oxopentanoate, 1.38 g (7.19 mmol) of 1-(3-dimethylaminopropyl)-3-ethylcarbodiimide hydrochloride and 0.073 g (0.6 mmol) of 4-dimethylaminopyridine are combined in 80 ml of dichloromethane and 20 ml of DMF and stirred at room temperature overnight. The reaction mixture is then poured into a mixture of semisaturated ammonium chloride solution and dichloromethane. The organic phase is separated off, washed successively with water, saturated sodium bicarbonate solution and saturated sodium chloride solution, dried over magnesium sulfate, filtered and concentrated. The residue is precipitated from dichloromethane using petroleum ether. The precipitate is filtered off with suction, washed with diethyl ether and dried under high vacuum. 4.44 g (92% of theory) of the protected intermediate remain.

LC-MS (Method 10): Rₜ=3.38 min; MS (ESIpos): m/z=605 (M+H)⁺.

250 mg (0.31 mmol) of the intermediate obtained are taken up in 8 ml of a saturated solution of hydrogen chloride in dichloromethane and allowed to stand at room temperature for 60 h. The precipitate formed is filtered off with suction, washed with diethyl ether and then dried under high vacuum. This gives 205 mg (96% of theory) of the title compound as colorless crystals.

HPLC (Method 7): Rₜ=5.2 min;

LC-MS (Method 11): Rₜ=1.79 min; MS (ESIpos): m/z=649 (M+H)⁺.

### Example 37

### 2-{4-[2-({[2-(4-Chlorophenyl)-1,3-thiazol-4-yl]methyl}thio)-3,5-dicyano-6-{[4-(dimethylamino)-butanoyl]amino}pyridin-4-yl]phenoxy}ethyl L-valinate bistrifluoracetate

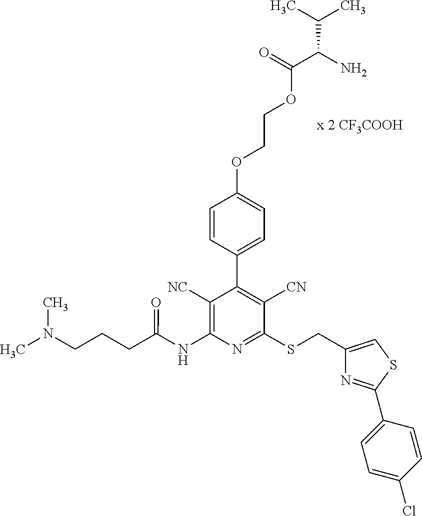

### Step a):

1 g (1.92 mmol) of 2-amino-6-({[2-(4-chlorophenyl)-1,3-thiazol-4-yl]methyl}thio)-4-[4-(2-hydroxy-ethoxy)phenyl]pyridine-3,5-dicarbonitrile, 0.460 g (2.11 mmol) of Boc-L-valine, 0.442 g (2.31 mmol) of 1-(3-dimethylaminopropyl)-3-ethylcarbodiimide hydrochloride and 0.023 g (0.19 mmol) of 4-dimethylaminopyridine are combined in 40 ml of dichloromethane and 10 ml of DMF and stirred at room temperature overnight. A clear solution is formed. The reaction mixture is then poured into a mixture of semisaturated ammonium chloride solution and dichloromethane. The organic phase is separated off, washed successively with water, saturated sodium bicarbonate solution and saturated sodium chloride solution, dried over magnesium sulfate, filtered and concentrated. The residue is purified by flash chromatography on silica gel using the mobile phase dichloromethane/ethyl acetate (gradient 10:1→7:1→5:1). The appropriate fractions are combined, and the solvent is removed under reduced pressure. After the residue has been dried under high vacuum, 0.85 g (62% of theory) of the protected intermediate remains.

### Step b):

200 mg (0.28 mmol) of the intermediate from Step a), 93 mg (0.56 mmol) of 4-(N,N-dimethyl-amino)butyric acid hydrochloride, 160 mg (0.84 mmol) of 1-(3-dimethylaminopropyl)-3-ethylcarbodiimide hydrochloride and 136 mg (1.1 mmol) of 4-dimethylaminopyridine are combined in 50 ml of dichloromethane and heated under reflux for 20 min. The reaction mixture is then concentrated, and the residue is purified by preparative HPLC (Method 6b). The appropriate fractions are combined and lyophilized from dioxane. After drying under high vacuum, 95 mg (41% of theory) of the protected bisacyl compound are obtained as a colorless foam.

LC-MS (Method 10): Rₜ=2.3 min; MS (ESIpos): m/z=832 (M+H)⁺.

### Step c):

50 mg (0.06 mmol) of the intermediate from Step b) are taken up in 10 ml of dichloromethane and 5 ml of anhydrous trifluoroacetic acid and stirred at room temperature for 1 h. The reaction mixture is then concentrated to dryness. The residue is taken up in water and lyophilized. This gives 51 mg (89% of theory) of the title compound as a colorless foam.

HPLC (Method 7): Rₜ=4.9 min;

LC-MS (Method 12): Rₜ=1.37 min; MS (ESIpos): m/z=732 (M+H)⁺.

### Example 38

### 2-{4-[2-Amino-6-({[2-(4-chlorophenyl)-1,3-thiazol-4-yl]methyl}sulfanyl)-3,5-dicyanopyridin-4-yl]-phenoxy}ethyl L-argininate trihydrochloride

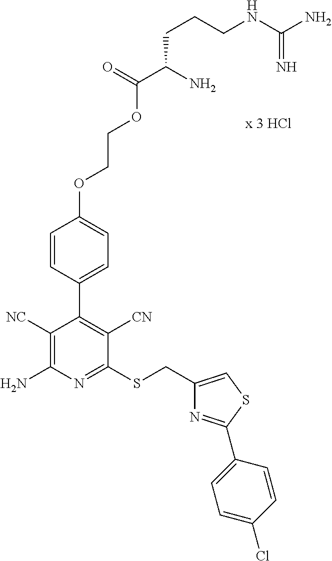

0.150 g (0.288 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 0.411 g (0.865 mmol) of N⁵-[N,N′-bis(tert-but-oxycarbonyl)carbamidoyl]-N²-(tert.-butoxycarbonyl)-L-ornithine, 0.083 g (0.433 mmol) of 1-(3-di-methylaminopropyl)-3-ethylcarbodiimide hydrochloride and 0.018 g (0.144 mmol) of 4-dimethylaminopyridine are combined in 30 ml of dichloromethane and 30 ml of DMF and stirred at room temperature for 3 days. The reaction mixture is then concentrated. The residue is taken up in dichloromethane and extracted successively with 5% strength citric acid, sodium bicarbonate solution and water. The organic phase is concentrated and the residue is purified by flash chromatography on silica gel using the mobile phase dichloromethane/ethyl acetate (4:1). The appropriate fractions are combined, and the solvent is removed under reduced pressure. After the residue has been dried under high vacuum, 93 mg (33% of theory) of the protected intermediate remain.

The intermediate obtained is taken up in 4 ml of dichloromethane and 2 ml of anhydrous trifluoroacetic acid, and the solution is stirred at room temperature for 1 h. The reaction mixture is then concentrated to dryness, and the residue two more times evaporated with acetonitrile. A 2 M solution of hydrogen chloride in diethyl ether is then added to the residue that remains. The precipitate formed is filtered off with suction, washed with diethyl ether and dried under high vacuum. This gives 51 mg (68% of theory) of the title compound as colorless crystals.

HPLC (Method 7): Rₜ=4.8 min;

LC-MS (Method 10): Rₜ=1.52 min; MS (ESIpos): m/z=676 (M+H)⁺.

### Example 39

### 2-{4-[2-Amino-6-({[2-(4-chlorophenyl)-1,3-thiazol-4-yl]methyl}sulfanyl)-3,5-dicyanopyridin-4-yl]-phenoxy}ethyl D-ornithinate dihydrochloride

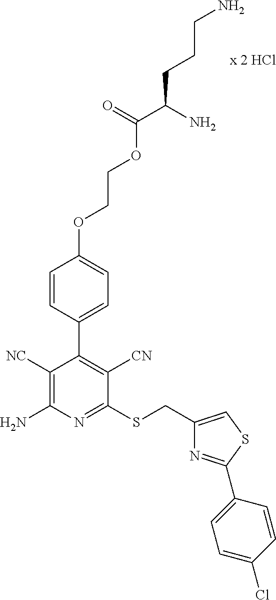

78 mg (0.15 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 150 mg (0.451 mmol) of N^{α},N^{δ}-di-Boc-D-ornithine, 37.5 mg (0.196 mmol) of 1-(3-dimethylaminopropyl)-3-ethylcarbodiimide hydrochloride and 9 mg (0.075 mmol) of 4-dimethylaminopyridine are combined in 15 ml of dichloromethane and 15 ml of DMF and stirred at room temperature overnight. Another 15 mg (0.045 mmol) of N^{α},N^{δ}-di-Boc-D-ornithine and 10 mg of 1-(3-dimethylaminopropyl)-3-ethylcarbodiimide hydrochloride are then added, and the reaction mixture is stirred at RT for another 16 h. The reaction mixture is then concentrated. The residue is taken up in dichloromethane and extracted successively with 5% strength citric acid, sodium bicarbonate solution and water. The organic phase is concentrated, and the residue is purified by flash chromatography on silica gel using the mobile phase dichloromethane/methanol/17% strength aqueous ammonia (15:0.5:0.05). The appropriate fractions are combined, and the solvent is removed under reduced pressure. After the residue has been dried under high vacuum, 43 mg (34% of theory) of the protected intermediate remain.

40 mg (0.048 mmol) of the intermediate obtained are taken up in 5 ml of dichloromethane and 1 ml of anhydrous trifluoroacetic acid, and the solution is stirred at room temperature for 30 min. The reaction mixture is then concentrated to dryness, and two more times the residue is evaporated with acetonitrile. A 2 M solution of hydrogen chloride in diethyl ether is then added to the residue that remains. The precipitate formed is separated off by decanting the supernatant, taken up in water, concentrated slightly and then lyophilized. This gives 32 mg (94% of theory) of the title compound.

HPLC (Method 7): Rₜ=4.8 min;

LC-MS (Method 10): Rₜ=1.54 min; MS (ESIpos): m/z=634 (M+H)⁺

¹H-NMR (400 MHz, DMSO-d₆): δ=1.6-2.0 (m, 4H), 2.8 (m, 2H), 4.1 (m, 1H), 4.35 (m, 2H), 4.55 (m, 2H), 4.65 (s, 2H), 7.12 (d, 2H), 7.48 (d, 2H), 7.57 (d, 2H), 7.92 (d, 2H), 7.95 (s, 1H), 8.0 (br. s, 2H), 8.55-8.65 (m, 2H).

### Example 40

### 2-{4-[2-Amino-6-({[2-(4-chlorophenyl)-1,3-thiazol-4-yl]methyl}sulfanyl)-3,5-dicyanopyridin-4-yl]-phenoxy}ethyl-3-amino L-alaninate dihydrochloride

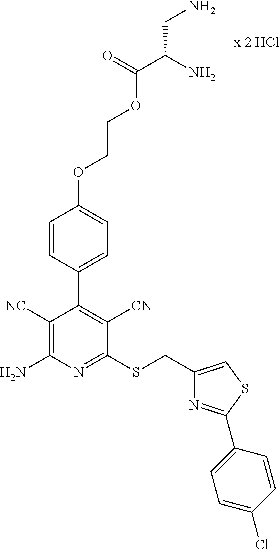

71 mg (0.137 mmol) of 2-amino-6-({[2-(4-chlorophenyl)-1,3-thiazol-4-yl]methyl}thio)-4-[4-(2-hydroxyethoxy)phenyl]pyridine-3,5-dicarbonitrile, 200 mg (0.412 mmol) of N-(tert-butoxycarbonyl)-3-[(tert-butoxycarbonyl)amino]-L-alanine dicyclohexylamine salt, 40 mg (0.206 mmol) of 1-(3-dimethylaminopropyl)-3-ethylcarbodiimide hydrochloride and 8.4 mg (0.069 mmol) of 4-dimethylaminopyridine are combined in 15 ml of dichloromethane and 15 ml of DMF and stirred at room temperature overnight. Another 20 mg (0.103 mmol) of 1-(3-dimethylamino-propyl)-3-ethylcarbodiimide hydrochloride and 4 mg (0.035 mmol) of 4-dimethylaminopyridine are then added, and the mixture is stirred at RT for another 16 h. The reaction mixture is then concentrated. The residue is taken up in dichloromethane and extracted successively with 5% strength citric acid, sodium bicarbonate solution and water. The organic phase is concentrated, and the residue is purified by flash chromatography on silica gel using the mobile phase dichloromethane/ethyl acetate (3:1). The appropriate fractions are combined, and the solvent is removed under reduced pressure. After the residue has been dried under high vacuum, 39 mg (35% of theory) of the protected intermediate remain.

The intermediate obtained is taken up in 5 ml of dichloromethane and 1 ml of anhydrous trifluoroacetic acid, and the solution is stirred at room temperature for 30 min. The reaction mixture is then concentrated to dryness, and two more times the residue is evaporated with acetonitrile. A 2 M solution of hydrogen chloride in diethyl ether is then added to the residue that remains. The precipitate formed is filtered off with suction, washed with diethyl ether and dried under high vacuum. This gives 26 mg (79% of theory) of the title compound as colorless crystals.

HPLC (Method 7): Rₜ=4.8 min;

LC-MS (Method 10): Rₜ=1.74 min; MS (ESIpos): m/z=606 (M+H)⁺.

### B. Determination of Solubility, Stability and Liberation Behavior

### a) Determination of the Solubility:

The test substance is suspended in water or dilute hydrochloric acid (pH 4) [Examples 1-21] or in 5% strength aqueous dextrose solution [Examples 22-40]. This suspension is shaken at room temperature for 24 h. After ultracentrifugation at 224 000 g for 30 min, the supernatant is diluted with DMSO and analyzed by HPLC. A two-point calibration plot of the test compound in DMSO is used for quantification.

HPLC method for acids:

Agilent 1100 with DAD (G1315A), quat. pump (G1311A), autosampler CTC HTS PAL, degasser (G1322A) and column thermostat (G1316A); column: Phenomenex Gemini C18, 5 μm, 50 mm×2 mm; temperature: 40° C.; eluent A: water/phosphoric acid pH 2, eluent B: acetonitrile; flow rate: 0.7 ml/min; gradient: 0-0.5 min 85% A, 15% B; ramp 0.5-3 min 10% A, 90% B; 3-3.5 min 10% A, 90% B; ramp 3.5-4 min 85% A, 15% B; 4-5 min 85% A, 15% B.

HPLC method for bases:

Agilent 1100 with DAD (G1315A), quat. pump (G1311A), autosampler CTC HTS PAL, degasser (G1322A) and column thermostat (G1316A); column: VDSoptilab Kromasil 100 C18, 3.5 μm, 60 mm×2.1 mm; temperature: 30° C.; eluent A: water+5 ml of perchloric acid/liter, eluent B: acetonitrile; flow rate: 0.75 ml/min; gradient: 0-0.5 min 98% A, 2% B; ramp 0.5-4.5 min 10% A, 90% B; 4.5-6 min 10% A, 90% B; ramp 6.5-6.7 min 98% A, 2% B; 6.7-7.5 min 98% A, 2% B.

The solubilities of representative exemplary embodiments in dilute hydrochloric acid (pH 4) are shown in Table 1:

TABLE 1

| Example No. | Solubility [mg/liter] |
|---|---|
| 6 | 90 |
| 9 | 50 |
| 10 | 8 |
| 11 | 70 |
| 15 | 240 |
| 18 | 60 |
| 19 | 57 |

The solubilities of representative exemplary embodiments in 5% strength aqueous dextrose solution are shown in Table 2:

TABLE 2

| Example No. | Solubility [mg/liter] |
|---|---|
| 22 | >500 |
| 24 | >500 |
| 25 | 730 |
| 26 | >500 |
| 31 | 4 |
| 33 | 195 |
| 37 | 160 |
| 38 | >500 |
| 39 | >500 |
| 40 | >500 |

No decomposition of the exemplary compounds in these solutions is observed.

The solubility of the underlying active substance [compound (A)] in dilute hydrochloric acid (pH 4) is determined in this test to be <1 mg/liter and that in 5% strength aqueous dextrose solution is determined to be <0.1 mg/liter.

### b) Stability in Buffer at Various pH Values:

0.3 mg of the test substance is weighed into a 2 ml HPLC vial and 0.5 ml of acetonitrile or acetonitrile/DMSO (9:1) is added. The substance is dissolved by putting the sample vessel in an ultrasonic bath for about 10 seconds. Then 0.5 ml of the respective (buffer) solution is added, and the sample is again treated in the ultrasonic bath.

### (Buffer) Solutions Employed:

pH 4: 1 liter of Millipore water is adjusted to pH 4.0 with 1 N hydrochloric acid;
pH 5: 0.096 mol of citric acid and 0.2 mol of sodium hydroxide ad 1 liter of water;
pH 7.4: 90.0 g of sodium chloride, 13.61 g of potassium dihydrogen phosphate and 83.35 g of 1 N sodium hydroxide solution are made up to 1 liter with water; this solution is then further diluted 1:10 with Millipore water.
pH 8: 0.013 mol of borax and 0.021 mol of hydrochloric acid ad 1 liter of water.

5 μl portions of the sample solution are analyzed by HPLC for their content of unchanged test substance, and of parent substance (A) produced, every hour over a period of 24 hours at 37° C. The percentage areas of the appropriate peaks are used for quantification.

HPLC method for Examples 1-21:

Agilent 1100 with DAD (G1314A), binary pump (G1312A), autosampler (G1329A), column oven (G1316A), thermostat (G1330A); column: Kromasil 100 C18, 60 mm×2.1 mm, 3.5 μm; column temperature: 30° C.; eluent A: water+5 ml of perchloric acid/liter, eluent B: acetonitrile; gradient: 0-1.0 min 98% A, 2% B; 1.0-9.0 min 2% A, 98% B; 9.0-13.0 min 2% A, 98% B; 13.0-13.5 min 98% A, 2% B; 13.5-15.0 min 98% A, 2% B; flow rate: 0.75 ml/min; UV detection: 210 nm.

HPLC method for Examples 22-40:

Agilent 1100 with DAD (G1314A), binary pump (G1312A), autosampler (G1329A), column oven (G1316A), thermostat (G1330A); column: Kromasil 100 C18, 125 mm×4.6 mm, 5 μm; column temperature: 30° C.; eluent A: water+5 ml of perchloric acid/liter, eluent B: acetonitrile; gradient: 0-2.0 min 90% A, 10% B; 2.0-18.0 min 64% A, 36% B; 18.0-20.0 min 64% A, 36% B; 20.0-21.0 min 10% A, 90% B; 21.0-23.0 min 90% A, 10% B; 23.0-26.0 min 90% A, 10% B; flow rate: 2.0 ml/min; UV detection: 294 nm.

The ratios of the peak areas (F) at the respective time points in relation to the peak areas at the starting time are shown in Table 3 for representative exemplary embodiments:

TABLE 3

| | | % test substance | % test substance |
|---|---|---|---|
| | | after 4 h | after 24 h |
| Example | | [F(t = 4 h) × 100/ | [F(t = 24 h) × 100/ |
| No. | pH | F(t = 0 h)] | F(t = 0 h)] |
| 1 | 4 | 100 | 100 |
| 1 | 8 | 100 | 100 |
| 5 | 4 | 100 | 100 |
| 5 | 8 | 94 | 68 |
| 14 | 4 | 100 | 100 |
| 14 | 8 | 96 | 76 |
| 15 | 4 | 93 | 91 |
| 15 | 8 | 90 | 76 |
| 16 | 4 | 100 | 100 |
| 16 | 8 | 100 | 100 |
| 22 | 4 | 100 | 100 |
| 22 | 7.4 | 0 | 0 |
| 23 | 4 | 100 | 99 |
| 23 | 7.4 | 100 | 96 |
| 24 | 4 | 100 | 99 |
| 24 | 5 | 96 | 74 |
| 25 | 4 | 100 | 100 |
| 25 | 7.4 | 41 | 4 |
| 26 | 4 | 100 | 100 |
| 26 | 7.4 | 11 | 2 |
| 28 | 4 | 100 | 100 |
| 28 | 7.4 | 100 | 100 |
| 34 | 4 | 100 | 97 |
| 34 | 7.4 | 100 | 97 |
| 35 | 4 | 100 | 100 |
| 35 | 7.4 | 100 | 98 |
| 37 | 4 | 100 | 100 |
| 37 | 7.4 | 97 | 84 |
| 38 | 4 | 100 | 100 |
| 38 | 7.4 | 100 | 94 |
| 39 | 4 | 100 | 100 |
| 39 | 7.4 | 0 | 0 |
| 40 | 4 | 99 | 94 |
| 40 | 7.4 | 90 | 53 |

In this test there is found to be a decrease in the content of test substance at the same time as an increase in the active ingredient compound (A).

### c) In Vitro Stability in Rat and Human Plasma:

1 mg of the test substance is weighed into a 2 ml HPLC vial, and 1.5 ml of DMSO and 1 ml of water are added. The substance is dissolved by placing the sample vessel in an ultrasonic bath for about 10 seconds. 0.5 ml of rat or human plasma at 37° C. is added to 0.5 ml of this solution. The sample is shaken, and about 10 μl are removed for a first analysis (time point t₀). 4-6 further aliquots are removed for quantification in the period up to 2 hours after the start of incubation. The sample is kept at 37° C. during the time of the test. Characterization and quantification take place by HPLC.

### HPLC Method:

Agilent 1100 with DAD (G1314A), binary pump (G1312A), autosampler (G1329A), column oven (G1316A), thermostat (G1330A); column: Kromasil 100 C18, 250 mm×4 mm, 5 μm; column temperature: 30° C.; eluent A: water+5 ml of perchloric acid/liter, eluent B: acetonitrile; gradient: 0-8.0 min 53% A, 47% B; 8.0-18.0 min 53% A, 47% B; 18.0-20.0 min 90% A, 10% B; 20.0-21.0 min 90% A, 10% B; 21.0-22.5 min 98% A, 2% B; 22.5-25.0 min 98% A, 2% B; flow rate: 2 ml/min; UV detection: 294 nm.

Table 4 indicates the respective times for representative exemplary embodiments at which 50% of the maximum possible amount of active ingredient compound (A) have been produced (t_{50% A}) after incubation with rat plasma. For the evaluation, the ratio of the peak areas at the individual time points compared with the starting time point is used in each case.

TABLE 4

| | t_{50% A} [min] |
|---|---|
| Example No. | in rat plasma |
| 22 | 0.5 |
| 23 | 9.0 |
| 24 | 0.5 |
| 25 | 55 |
| 34 | >120 |
| 35 | >120 |
| 36 | 4.0 |
| 37 | >120 |
| 38 | 0.5 |
| 39 | 0.5 |
| 40 | 0.5 |

d) i.v. Pharmacokinetics in Wistar Rats:

On the day before administration of the substance, a catheter for obtaining blood is implanted in the jugular vein of the experimental animals (male Wistar rats, body weight 200-250 g) under Isofluran® anesthesia.

On the day of the experiment, a defined dose of the test substance is administered as solution into the tail vein using a Hamilton® glass syringe (bolus administration, duration of administration <10 s). Blood samples (8-12 time points) are taken through the catheter sequentially over the course of 24 h after administration of the substance. Plasma is obtained by centrifuging the samples in heparinized tubes. Acetonitrile is added to a defined plasma volume per time point to precipitate proteins. After centrifugation, test substance and, where appropriate, known cleavage products of the test substance in the supernatant are determined quantitatively using a suitable LC/MS-MS method.

The measured plasma concentrations are used to calculate pharmacokinetic parameters of the test substance and of the active ingredient compound (A) liberated therefrom, such as AUC, Cₘₐₓ, T_{1/2} (half-life) and CL (clearance).

After i.v. administration of the compound from Example 1, from Example 5, from Example 15 and from Example 22, these substances were no longer detectable in plasma even at the first measurement point. Only the active ingredient (A) was detectable up to the 24-hour time point too.

### e) Oral Pharmacokinetics in Wistar Rats:

On the day before administration of the substance, a catheter for obtaining blood is implanted in the jugular vein of the experimental animals (male Wistar rats, body weight 200-250 g) under Isofluran® anesthesia.

On the day of the experiment, a defined dose of the test substance is administered as solution into the stomach by gavage. Blood samples (8-12 time points) are taken through the catheter sequentially over the course of 24 h after administration of the substance. Plasma is obtained by centrifuging the samples in heparinized tubes. Acetonitrile is added to a defined plasma volume per time point to precipitate proteins. After centrifugation, test substance and, where appropriate, known cleavage products of the test substance in the supernatant are determined quantitatively using a suitable LC/MS-MS method.

The measured plasma concentrations are used to calculate pharmacokinetic parameters of the test substance and of the active ingredient compound (A) liberated therefrom, such as AUC, Cₘₐₓ, T_{1/2} (half-life).

After oral administration of the compound from Example 5, from Example 15, from Example 19 and from Example 22, these substances were no longer detectable in plasma even at the first measurement point. Only the active ingredient (A) was detectable up to the 24-hour time point too.

### f) Determination of the Influence on the Heart Rate of Anesthetized Rats:

Male Wistar rats with a body weight above 250 g are employed. In the night before the experiment, the animals receive no feed but still have free access to drinking water. Preparation and investigations are carried out under Trapanal® anesthesia (100 mg/kg i.p.). Injection and infusion take place through a catheter in the jugular vein, and the blood pressure is recorded via a catheter in the femoral artery (transducer: Braun, Melsungen). After the preparation, the animals are connected to a continuous infusion of physiological saline solution to compensate fluid losses. Test substance or placebo solution are administered intravenously as bolus after an equilibration time of about 1 h. Heart rate and arterial blood pressure are recorded during the equilibration and over a period of at least 30 min after the bolus injection with the aid of a digital evaluation program.

Table 5 lists the maximum heart rate decrease in the first 30 min after an i.v. bolus of 100 μg/kg of the active substance (A) or of equivalent dosages of representative exemplary embodiments:

TABLE 5

| Example | Heart rate decrease |
|---|---|
| No. | [%] |
| A | 24 |
| 1 | 16 |
| 5 | 21 |
| 15 | 21 |
| 19 | 18 |
| 22 | 13 |

### C. Exemplary Embodiments of Pharmaceutical Compositions

The compounds of the invention can, for example, be converted into pharmaceutical preparations in the following ways:

### Tablet:

### Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg. Diameter 8 mm, radius of curvature 12 mm.

### Production:

The mixture of the compound of the invention, lactose and starch is granulated with a 5% strength solution (m/m) of PVP in water. The granules are dried and then mixed with the magnesium stearate for 5 min. This mixture is compressed with a conventional tablet press (see above for format of the tablet). As guideline, a compressive force of 15 kN is used for the compression.

### Oral Suspension:

### Composition:

1000 mg of the compound of the invention, 1000 mg of ethanol (96%), 400 mg of Rhodigel® (xanthan gum from FMC, Pennsylvania, USA) and 99 g of water.

10 ml of oral suspension are equivalent to a single dose of 100 mg of the compound of the invention.

### Production:

The Rhodigel is suspended in ethanol, and the compound of the invention is added to the suspension. The water is added while stirring. The mixture is stirred for about 6 hours until the swelling of the Rhodigel is complete.

### Oral Solution:

### Composition:

500 mg of the compound of the invention, 2.5 g of polysorbate and 97 g of polyethylene glycol 400. A single dose of 100 mg of the compound of the invention corresponds to 20 g oral solution.

### Production:

The compound of the invention is suspended in the mixture of polyethylene glycol and polysorbate with stirring. The stirring is continued until the compound of the invention has completely dissolved.

i.v. Solution:

The compound of the invention is dissolved in a concentration below the saturation solubility in a physiologically tolerated solvent (e.g. isotonic saline solution, 5% glucose solution and/or 30% PEG 400 solution, in each case adjusted to a pH of 3-5). The solution is optionally filtered sterile and/or dispensed into sterile and pyrogen-free injection containers.

What is claimed is:

1. A compound of the formula (I) 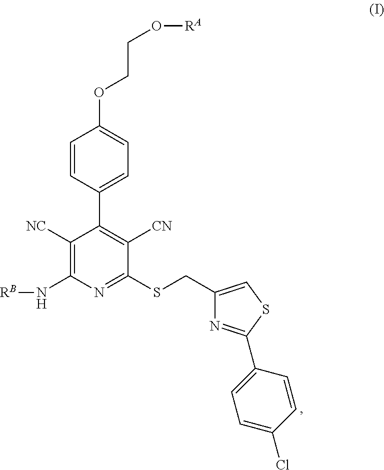
in which
R^{A} is a group of the formula 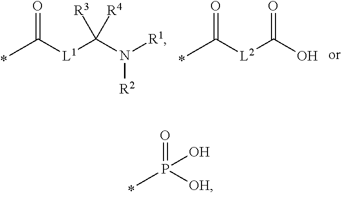
in which
* means the point of linkage to the O atom;
L¹ is a bond, —CH₂—, or —CH₂CH₂—;
R¹ and R² are identical or different and are independently of one another hydrogen or (C₁-C₄)-alkyl that may be substituted by hydroxyl, (C₁-C₄)-alkoxy, amino, mono-(C₁-C₄)-alkylamino, or di-(C₁-C₄)-alkylamino,
or
R¹ and R² are attached to one another and, together with the nitrogen atom to which they are attached, form a 5- or 6-membered saturated heterocycle that may contain a further ring heteroatom from the group consisting of N and O, and may be mono- or disubstituted by identical or different substituents from the group consisting of (C₁-C₄)-alkyl, amino, hydroxyl, and (C₁-C₄)-alkoxy;
R³ is hydrogen or the side group of a natural α-amino acid or its homologs or isomers,
or
R³ is attached to R¹ and the two, together with the atoms to which they are attached, form a 5- or 6-membered saturated heterocycle that may be mono- or disubstituted by identical or different substituents from the group consisting of (C₁-C₄)-alkyl, amino, hydroxyl, and (C₁-C₄)-alkoxy;
R⁴ is hydrogen or methyl;
L² is a bond or straight-chain (C₁-C₆)-alkanediyl or (C₂-C₆)-alkenediyl that may be substituted up to four times by identical or different radicals selected from the group consisting of (C₁-C₄)-alkyl, hydroxyl, (C₁-C₄)-alkoxy, amino, mono-(C₁-C₄)-alkylamino, and di-(C₁-C₄)-alkylamino,
where (C₁-C₄)-alkyl for its part may be substituted by hydroxyl, (C₁-C₄)-alkoxy, amino, mono-(C₁-C₄)-alkylamino, or di-(C₁-C₄)-alkylamino, and
two of the (C₁-C₄)-alkyl radicals mentioned may be attached to one another and together with the carbon atom(s) to which they are attached form a 3- to 6-membered saturated carbocycle that may be substituted by amino, hydroxyl or (C₁-C₄)-alkoxy; and
R^{B} is hydrogen or a group of the formula 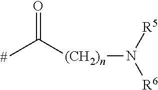
in which
# means the point of linkage to the N atom;
n is the number 1, 2, 3, or 4;
and
R⁵ and R⁶ are independently of one another hydrogen or (C₁-C₄)-alkyl,
or a pharmaceutically acceptable salt thereof.

2. The compound according to claim 1, wherein
R^{A} is a group of the formula 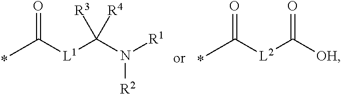
in which
* means the point of linkage to the O atom;
L¹ is a bond, —CH₂—, or —CH₂CH₂—;
R¹ and R² are independently of one another hydrogen or methyl,
or
R¹ and R² are attached to one another and, together with the nitrogen atom to which they are attached, form a pyrrolidino, piperidino, or morpholino ring;
R³ is hydrogen, methyl, propan-2-yl, propan-1-yl, butan-1-yl, benzyl, imidazol-4-yl-methyl, hydroxymethyl, 1-hydroxyethyl, 2-carboxyethyl, 4-aminobutan-1-yl, 3-amino-propan-1-yl, 2-aminoethyl, aminomethyl, or 3-guanidinopropan-1-yl,
or
R³ is attached to R¹ and the two, together with the atoms to which they are attached, form a pyrrolidine or piperidine ring;
R⁴ is hydrogen;
L² is methylene, 1,2-ethylene, 1,3-propylene, or ethene-1,2-diyl, wherein which 1,2-ethylene and 1,3-propylene may each be substituted by amino; and
R^{B} is hydrogen or a group of the formula 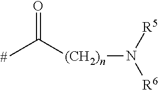
in which
# means the point of linkage to the N atom;
n is the number 1, 2, or 3; and
R⁵ and R⁶ independently of one another are hydrogen or methyl.

3. The compound according to claim 1, wherein
R^{A} is a group of the formula 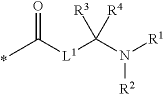
in which
* means the point of linkage to the O atom;
L¹ is a bond;
R¹ and R² are independently of one another hydrogen or methyl;
R³ is hydrogen, methyl, propan-2-yl, propan-1-yl, butan-1-yl, imidazol-4-ylmethyl, hydroxymethyl, 4-aminobutan-1-yl, 3-aminopropan-1-yl, 2-aminoethyl, aminomethyl, or 3-guanidinopropan-1-yl;
R⁴ is hydrogen; and
R^{B} is hydrogen.

4. A process for preparing a compound of the formula (I) 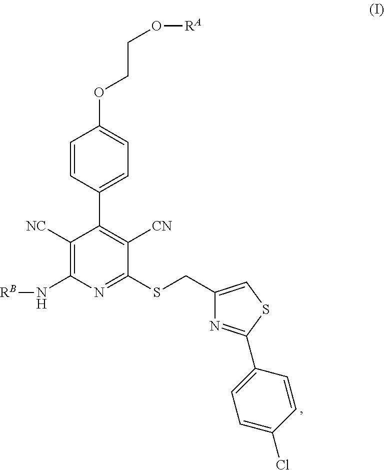
in which
R^{A} is a group of the formula 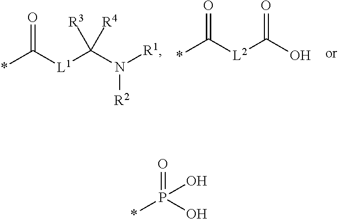
in which
* means the point of linkage to the O atom;
L¹ is a bond, —CH₂— or —CH₂CH₂—;
R¹ and R² are identical or different and are independently of one another hydrogen or (C₁-C₄)-alkyl that may be substituted by hydroxyl, (C₁-C₄)-alkoxy, amino, mono-(C₁-C₄)-alkylamino or di-(C₁-C₄)-alkylamino,
or
R¹ and R² are attached to one another and, together with the nitrogen atom to which they are attached, form a 5- or 6-membered saturated heterocycle that may contain a further ring heteroatom from the group consisting of N and O, and may be mono- or disubstituted by identical or different substituents from the group consisting of (C₁-C₄)-alkyl, amino, hydroxyl, and (C₁-C₄)-alkoxy;
R³ is hydrogen or the side group of a natural α-amino acid or its homologs or isomers
or
R³ is attached to R¹ and the two, together with the atoms to which they are attached, form a 5- or 6-membered saturated heterocycle that may be mono- or disubstituted by identical or different substituents from the group consisting of (C₁-C₄)-alkyl, amino, hydroxyl, and (C₁-C₄)-alkoxy;
R⁴ is hydrogen or methyl;
L² is a bond or straight-chain (C₁-C₆)-alkanediyl or (C₂-C₆)-alkenediyl that may be substituted up to four times by identical or different radicals selected from the group consisting of (C₁-C₄)-alkyl, hydroxyl, (C₁-C₄)-alkoxy, amino, mono-(C₁-C₄)-alkylamino, and di-(C₁-C₄)-alkylamino,
where (C₁-C₄)-alkyl for its part may be substituted by hydroxyl, (C₁-C₄)-alkoxy, amino, mono-(C₁-C₄)-alkylamino, or di-(C₁-C₄)-alkylamino,
and
two of the (C₁-C₄)-alkyl radicals mentioned may be attached to one another and, together with the carbon atom(s) to which they are attached, form a 3- to 6-membered saturated carbocycle that may be substituted by amino, hydroxyl or (C₁-C₄)-alkoxy; and
R^{B} is hydrogen,
or a pharmaceutically acceptable salt thereof,
the process comprising at least one of the steps of:
[A] reacting the compound (A) 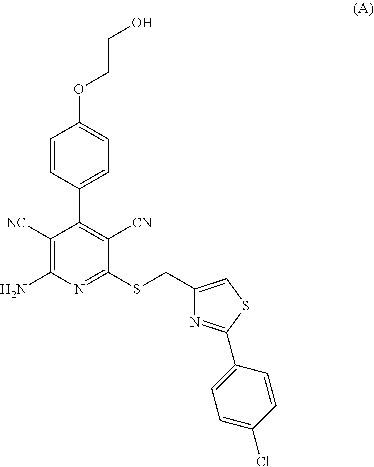
in an inert solvent in the presence of a base with phosphoryl chloride and subsequently heating with water to convert the compound (A) into the compound of the formula (I-A) 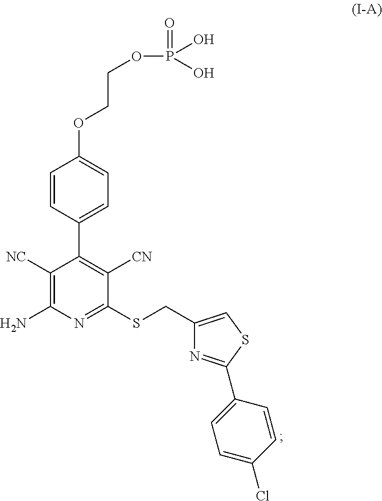
or
[B] coupling the compound (A) in an inert solvent with a compound of the formula (II) 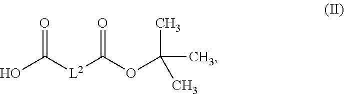
in which L² has the meaning indicated in claim 1,
with activation of the carboxyl group in formula (II) to give a compound of the formula (III) 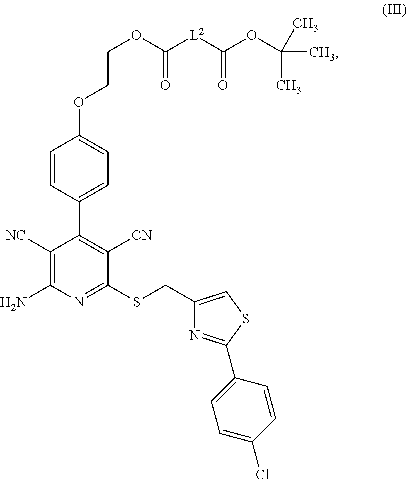
in which L² has the meaning indicated above,
cleaving the tert-butyl ester grouping with the aid of an acid to give a compound of the formula (I-B) 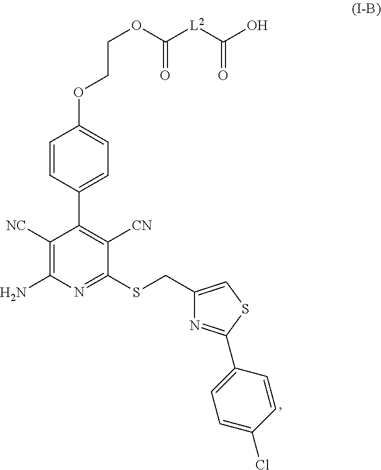
in which L² has the meaning indicated above;
or
[C] coupling the compound (A) in an inert solvent with a compound of the formula (IV) 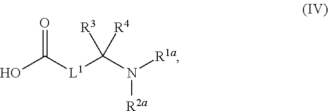
in which L¹, R³, and R⁴ have the meanings indicated in claim 1, and R^{1a} and R^{2a} are identical or different and have the meanings indicated in claim 1 for R¹ and R², respectively, or are temporary amino protective groups, with activation of the carboxyl group in formula (IV) to give a compound of the formula (V) 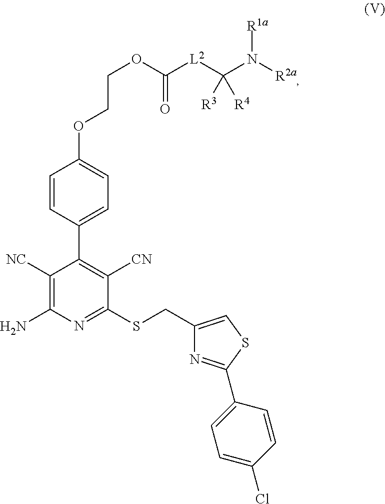
in which L¹, R^{1a}, R^{2a}, R³, and R⁴ have the meanings indicated above,
and removing any protective groups present to give a compound of the formula (I-C) 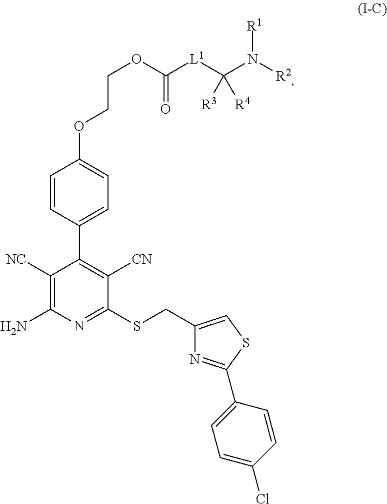
in which L¹, R¹, R², R³, and R⁴ have the meanings indicated in claim 1,
wherein the resulting compounds of the formulae (I-A), (I-B) and (I-C), respectively, are converted where appropriate with the appropriate (i) solvents and (ii) acids or bases into the salts thereof.

5-6. (canceled)

7. A pharmaceutical composition comprising a compound according to claim 1 and a pharmaceutically acceptable excipient.

8-9. (canceled)

10. A method for the treatment or prophylaxis of cardiovascular disorders in humans and animals comprising the step of administering to a human or animal in need thereof a compound of the formula (I) 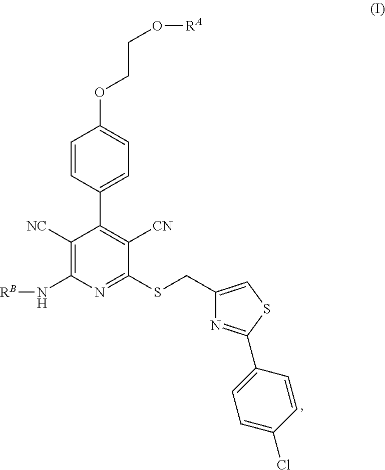
in which
R^{A} is a group of the formula 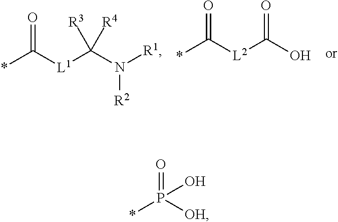
in that
* means the point of linkage to the O atom;
L¹ is a bond, —CH₂— or —CH₂CH₂—;
R¹ and R² are identical or different and are independently of one another hydrogen or (C₁-C₄)-alkyl that may be substituted by hydroxyl, (C₁-C₄)-alkoxy, amino, mono-(C₁-C₄)-alkylamino, or di-(C₁-C₄)-alkylamino,
or
R¹ and R² are attached to one another and, together with the nitrogen atom to which they are attached, form a 5- or 6-membered saturated heterocycle that may contain a further ring heteroatom from the group consisting of N and O, and may be mono- or disubstituted by identical or different substituents from the group consisting of (C₁-C₄)-alkyl, amino, hydroxyl, and (C₁-C₄)-alkoxy;
R³ is hydrogen or the side group of a natural α-amino acid or its homologs or isomers
or
R³ is attached to R¹ and the two, together with the atoms to which they are attached, form a 5- or 6-membered saturated heterocycle that may be mono- or disubstituted by identical or different substituents from the group consisting of (C₁-C₄)-alkyl, amino, hydroxyl, and (C₁-C₄)-alkoxy;
R⁴ is hydrogen or methyl;
L² is a bond straight-chain (C₁-C₆)-alkanediyl or (C₂-C₆)-alkenediyl that may be substituted up to four times by identical or different radicals selected from the group consisting of (C₁-C₄)-alkyl, hydroxyl, (C₁-C₄)-alkoxy, amino, mono-(C₁-C₄)-alkylamino, and di-(C₁-C₄)-alkylamino,
where (C₁-C₄)-alkyl for its part may be substituted by hydroxyl, (C₁-C₄)-alkoxy, amino, mono-(C₁-C₄)-alkylamino, or di-(C₁-C₄)-alkylamino,
and
two of the (C₁-C₄)-alkyl radicals mentioned may be attached to one another and, together with the carbon atom(s) to which they are attached, form a 3- to 6-membered saturated carbocycle that may be substituted by amino, hydroxyl, or (C₁-C₄)-alkoxy; and
R^{B} is hydrogen or a group of the formula 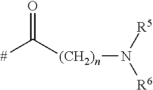
in which
# means the point of linkage to the N atom;
n is the number 1, 2, 3, or 4;
and
R⁵ and R⁶ are independently of one another hydrogen or (C₁-C₄)-alkyl,
or a pharmaceutically acceptable salt thereof.

11. A pharmaceutical composition comprising a compound according to claim 2 and a pharmaceutically acceptable excipient.

12. A pharmaceutical composition comprising a compound according to claim 3 and a pharmaceutically acceptable excipient.

13. The method of claim 10, wherein, for the compound of the formula (I), R^{A} is a group of the formula 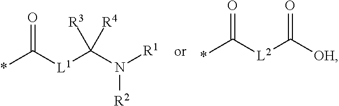
in which
* means the point of linkage to the O atom;
L¹ is a bond, —CH₂—, or —CH₂CH₂—;
R¹ and R² are independently of one another hydrogen or methyl,
or
R¹ and R² are attached to one another and, together with the nitrogen atom to which they are attached, form a pyrrolidino, piperidino, or morpholino ring;
R³ is hydrogen, methyl, propan-2-yl, propan-1-yl, butan-1-yl, benzyl, imidazol-4-yl-methyl, hydroxymethyl, 1-hydroxyethyl, 2-carboxyethyl, 4-aminobutan-1-yl, 3-amino-propan-1-yl, 2-aminoethyl, aminomethyl, or 3-guanidinopropan-1-yl,
or
R³ is attached to R¹ and the two, together with the atoms to which they are attached, form a pyrrolidine or piperidine ring;
R⁴ is hydrogen;
L² is methylene, 1,2-ethylene, 1,3-propylene, or ethene-1,2-diyl, wherein the 1,2-ethylene and 1,3-propylene may each be substituted by amino;
and
R^{B} is hydrogen or a group of the formula 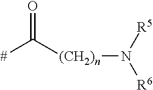
in which
# means the point of linkage to the N atom;
n is the number 1, 2, or 3;
and
R⁵ and R⁶ independently of one another are hydrogen or methyl.

14. The method of claim 10, wherein, for the compound of the formula (I), R^{A} is a group of the formula 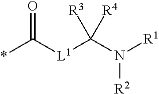
in which
* means the point of linkage to the O atom;
L¹ is a bond;
R¹ and R² are independently of one another hydrogen or methyl;
R³ is hydrogen, methyl, propan-2-yl, propan-1-yl, butan-1-yl, imidazol-4-ylmethyl, hydroxymethyl, 4-aminobutan-1-yl, 3-aminopropan-1-yl, 2-aminoethyl, aminomethyl, or 3-guanidinopropan-1-yl;
R⁴ is hydrogen; and
R^{B} is hydrogen.
